(19) United States
(12) Patent Application Publication
(10) Pub. No.: US 2020/0384130 A1
(43) Pub. Date: Dec. 10, 2020

(54) B-CELL MATURATION ANTIGEN (BCMA)-DIRECTED NANOPARTICLES

(71) Applicant: DANA-FARBER CANCER INSTITUTE, INC., Boston, MA (US)

(72) Inventors: Alexandre DETAPPE, Boston, MA (US); Irene GHOBRIAL, Boston, MA (US); Mairead REIDY, Brookline, MA (US); Peter GHOROGHCHIAN, Boston, MA (US)

(73) Assignee: DANA-FARBER CANCER INSTITUTE, INC., Boston, MA (US)

(21) Appl. No.: 16/620,252

(22) PCT Filed: Jun. 13, 2018
(86) PCT No.: PCT/US2018/037284

§ 371 Date: Dec. 6, 2019

Related U.S. Application Data
(60) Provisional application No. 62/519,643, filed on Jun. 14, 2017.
(60) Provisional application No. 62/524,952, filed on Jun. 26, 2017.

(51) Int. Cl. A61K 49/18; A61K 47/69; C07K 16/28

(52) U.S. Cl. CPC A61K 49/1881; B82Y 5/00; C07K 16/2878; A61K 47/6931

(57) ABSTRACT

The present invention relates to compositions comprising B-cell maturation antigen-directed nanoparticles and methods for using the same.

### RELATED APPLICATIONS

This application claims the benefit of priority under 35 U.S.C. § 119(e) to U.S. Provisional Application No. 62/519,643, filed Jun. 14, 2017, and to U.S. Provisional Application No. 62/524,952, filed Jun. 26, 2017, each of which is incorporated herein by reference in its entirety.

### FIELD OF THE INVENTION

The invention relates generally to B-cell maturation antigen (BCMA)-targeted compositions.

### BACKGROUND OF THE INVENTION

Effective diagnosis of minimal residual disease (MRD) plays a critical role in cancer control and treatment response monitoring. The level of MRD for multiple myeloma (MM) patients is directly linked to both the extent of response to treatment and long-term outcomes. Prior to the invention described herein, there was a pressing need to develop improved imaging agents that allow for improved detection and treatment of MM, particularly for the presence of MRD in MM patients.

### BRIEF SUMMARY OF THE INVENTION

The invention relates to B-cell maturation antigen (BCMA)-targeted compositions, including those comprising BCMA-targeted nanoparticles possessing enhanced imaging effects as compared to existing nanoparticles, as well as methods for the study, diagnosis, and treatment of traits, diseases and conditions for which BCMA-targeted compositions are useful (e.g., multiple myeloma).

The present invention is based, at least in part, upon the identification of non-invasive imaging compositions and techniques that specifically target cell-surface receptors of plasma cells. Such compositions and techniques are particularly useful for detecting MRD (via biomarker detection) and also allow for a quick and painless evaluation of treatment progress and/or outcome, while also allowing the user to account for spatial heterogeneity typical of the disease, as such spatial heterogeneity is refractory to assessment by, e.g., bone marrow sampling, flow cytometry and/or molecular study. Described herein is the identification of a cell surface targeting composition that includes silica-based gadolinium nanoparticles (NPs) that are conjugated to a monoclonal anti-B cell maturation antigen (BCMA). The NP is used for in vivo magnetic resonance imaging of the BCMA cell surface receptor, as a biomarker useful for monitoring a therapeutic response to MM treatment in a cell, tissue or subject, and for assessing the presence of minimal residual disease MRD in a cell, tissue and/or MM subject.

Specifically, described herein is a targeted nanoparticle conjugate comprising a nanoparticle; a linker; and an anti-BCMAantibody, e.g., an anti-BCMA-monoclonal antibody. In certain embodiments, the nanoparticle of the targeted nanoparticle conjugate is less than 10 nm in size, e.g., less than 9 nm, less than 8 nm, less than 7 nm, less than 6 nm, less than 5 nm, less than 4 nm, less than 3 nm, less than 2 nm, or less than 1 nm. An exemplary nanoparticle comprises a gadolinium nanoparticle. For example, the nanoparticle comprises a silica-based gadolinium nanoparticle (SiGdNP). In some cases, the nanoparticle can range up to 30 nm or more in size (e.g., 50 nm or less, 40 nm or less, 35 nm or less, 34 nm or less, 33 nm or less, 32 nm or less, 31 nm or less, 30 nm or less, 10-50 nm, 15-45 nm, 20-40 nm, 25-35 nm, 20-30 nm, etc.), for example, in embodiments in which the conjugate includes a polymer brush nanoparticle or a nanoparticle including clustered regularly interspaced short palindromic repeats (CRISPR) machinery (i.e. sgRNA guides and/or Cas9 mRNA) agents. It is believed that the larger nanoparticles degrade, thereby minimizing toxicity.

In one aspect, the nanoparticle comprises a polymer nanoparticle. Optionally, the targeted nanoparticle conjugate further comprises a drug. Alternatively, the nanoparticle comprises an inorganic nanoparticle. In some cases, the targeted nanoparticle conjugate is approximately 6-15 nm in size, optionally about 8-12 nm in size, optionally wherein the size of the targeted nanoparticle conjugate is stable over time, optionally wherein the size of the targeted nanoparticle conjugate is stable over a period of 15 min or more, 30 min or more, an hour or more, two hours or more four hours or more, eight hours or more, a day or more, two days or more, three days or more, or a week or longer. In other embodiments, the targeted nanoparticle conjugate is approximately 15-60 nm in size, optionally about 20-50 nm in size, optionally about 30-50 nm in size, optionally about 35-45 nm in size, optionally 40 nm in size or more, optionally wherein the size of the targeted nanoparticle conjugate is stable over time, optionally wherein the size of the targeted nanoparticle conjugate is stable over a period of 15 min or more, 30 min or more, an hour or more, two hours or more four hours or more, eight hours or more, a day or more, two days or more, three days or more, or a week or longer.

Exemplary linkers include homobifunctional amine-amine linker (N-Hydroxysuccinimide (NHS)-to-NHS) linker, heterobifunctional amine-to-sulfydryl (NHS-to-haloacetyl, NHS-maleimide, NHS-pyridyldithiol) linker. Without wishing to be bound by theory, in certain embodiments, an NHS linker conjugates to a polymer and/or NP of the disclosure, then the NHS linker also conjugates to the antibody of the disclosure, with this latter attachment occurring via, e.g., a NHS, thiol, maleimide or haloacetyl. A suitable anti-BCMA antibody includes a monoclonal antibody or fragment thereof. For example, the anti-BCMA antibody comprises a human monoclonal antibody or fragment thereof. Exemplary anti-BCMA antibody fragments include a Fv, a Fab, a Fab′, a Fab′-SH, a F(ab′)2, a diabody, a linear antibody, a single-chain antibody molecule (e.g., scFv) and a multispecific antibody formed from antibody fragments.

In some cases, the anti-BCMA antibody is labeled. For example, the anti-BCMA antibody is labeled with peridinin chlorophyll protein complex (PerCP)/Cy5.5.

In one aspect, the targeted nanoparticle conjugate comprises a nanoparticle core decorated with free NHS groups. Optionally, the NHS groups are conjugated on the surface of the anti-BCMA antibody via a bissulfosuccinimidyl suberate crosslinker.

In some cases, the nanoparticle conjugate further comprises a drug moiety. For example, the drug moiety is, an anti-CS1 antibody or drug (e.g., Elotuzamab) or an anti-CD38 antibody or drug (e.g., Daratumumab).

Also provided is a formulation comprising the targeted nanoparticle conjugate described herein. Preferably, the targeted nanoparticle conjugate is present at a dose equivalent of 0.1-1 mg/g of SiGdNP, e.g., about 0.2 mg/g, 0.3 mg/g, 0.4 mg/g, 0.5 mg/g, 0.6 mg/g, 0.7 mg/g, 0.8 mg/g, or 0.9 mg/g of SiGdNP. For example, the targeted nanoparticle conjugate is present at a dose equivalent of about 0.25 mg/g of SiGdNP.

Also provided is a pharmaceutical composition comprising the targeted nanoparticle conjugate described herein and a pharmaceutically acceptable carrier.

Methods for detecting the presence and/or localization of multiple myeloma (MM) and/or minimal residual disease (MRD) in a subject are carried out by administering the targeted nanoparticle conjugate described herein to the subject and detecting the presence and/or localization of the targeted nanoparticle conjugate in the subject, thereby detecting the presence and/or localization of MM and/or MRD in the subject. In certain embodiments, the step of administering is performed by injection, optionally by intravenous or intraperitoneal injection.

For example, the step of detecting comprises utilization of a magnetic resonance imaging (MRI) scan. In one aspect, the targeted nanoparticle conjugate acts as an imaging biomarker for the detection of MM cells and/or MRD in the subject. In some cases, the targeted nanoparticle conjugate, e.g., the BCMA-targeted NP, provides contrast that is improved by at least 5-fold, optionally by at least 10-fold, optionally about 12-fold or more as compared to an appropriate non-targeted NP control, e.g., a NP that is not targeted to BCMA. For example, the targeted nanoparticle conjugate provides contrast that is improved by at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, or at least 20 fold or more as compared to an appropriate non-targeted NP control. In some cases, the signal-to-noise ratio (SNR) and normalized SNR are calculated according to equations (1) and (2): (1) SNR=intensity/noise; (2) Normalized SNR(i)=SNR(i)/SNR_{baseline}. Without wishing to be bound by theory, the enhanced imaging attributes of the targeted NPs of the instant disclosure are believed to be attributable to the robust cell-targeting efficacies of the anti-BCMA antibodies as described herein. While untargeted and/or passive targeting NPs are mostly directed to tumor cells by neoangiogenesis, such untargeted and/or passive targeting NPs do not target plasma cells, thereby creating “noise” (e.g., more diffuse imaging signal) within the healthy tissues of a subject.

In certain embodiments, the targeted nanoparticle conjugate possesses a MRI detection threshold for MRD of 100,000 or less plasma cells per subject, optionally 50,000 or less plasma cells per subject, optionally 30,000 or less plasma cells per subject, optionally 20,000 or less plasma cells per subject, optionally 10,000 or less plasma cells per subject, optionally 8,000 or less plasma cells per subject, optionally 6,000 or less plasma cells per subject, optionally 5,000 or less plasma cells per subject, optionally 4,000 or less plasma cells per subject, optionally 3,000 or less plasma cells per subject, optionally about 2,200 plasma cells per subject—e.g., optionally 2,200±450 plasma cells per subject (optionally, where the subject is a mouse).

In some cases, the step of detecting is performed within approximately 1 hour of the step of administering the targeted nanoparticle conjugate, optionally within approximately 30 minutes of the step of administering the targeted nanoparticle conjugate. In other cases, the step of detecting is performed within 5 minutes, within 10 minutes, within 15 minutes, within 20 minutes, within 25 minutes, within 30 minutes, within 35 minutes, within 40 minutes, within 45 minutes, within 50 minutes, within 55 minutes, within 60 minutes, within 65 minutes, within 70 minutes, within 75 minutes, within 80 minutes, within 85 minutes, or within 90 minutes of the step of administering the targeted nanoparticle conjugate.

In certain other embodiments, the step of detecting is performed within approximately 12-48 hours after the step of administering the targeted nanoparticle conjugate, optionally within approximately 36 hours of the step of administering the targeted nanoparticle conjugate, optionally within about 35 hours, within about 34 hours, within about 33 hours, within about 32 hours, within about 31 hours, within about 30 hours, within about 29 hours, within about 28 hours, within about 27 hours, within about 26 hours, within about 25 hours, within about 24 hours, within about 23 hours, within about 22 hours, within about 21 hours, within about 20 hours, within about 19 hours, within about 18 hours, within about 17 hours, within about 16 hours, within about 15 hours, within about 14 hours, within about 13 hours, within about 12 hours, within about 11 hours, within about 10 hours, within about 9 hours, within about 8 hours, within about 7 hours, within about 6 hours, within about 5 hours, within about 4 hours, within about 3 hours, or within about 2 hours, of the step of administering the targeted nanoparticle conjugate.

In one aspect, the targeted nanoparticle conjugate binds approximately 70% of MM cells at 30 minutes after the step of administering the targeted nanoparticle conjugate. In another aspect, the targeted nanoparticle conjugate binds at least about 50%, at least about 55%, at least about 60%, at least about 65%, at least about 70%, at least about 75%, at least about 80%, at least about 85%, at least about 90%, at least about 95%, or about 100% of MM cells.

Optionally, the targeted nanoparticle conjugate is detected in spine, femur other bone, and/or spleen.

Preferably, herein tumor uptake of the targeted nanoparticle conjugate is enhanced relative to an appropriate control non-targeted nanoparticle.

In one aspect, detecting the presence and/or localization of MM and/or MRD in the subject is used to assess a MM therapy. For example, the therapy comprises administration of an anti-CS1 antibody or drug (e.g., Elotuzamab) or an anti-CD38 antibody or drug (e.g., Daratumumab). In another example, the targeted nanoparticle conjugate is administered in combination with the MM therapy.

Preferably, the subject is human. Alternatively, the subject is murine. For example, the subject is a MRD model mouse. Optionally, the MRD model mouse is induced by administration of Bortezomib and Melphalan. In one aspect, xenograft-derived MM is detected in severe combined immune deficiency (SCID)/beige mice.

In some cases, detecting the presence and/or localization of MM and/or MRD in the subject comprises detecting disease progression from monoclonal gammopathy of undetermined significance (MGUS) to smoldering multiple myeloma (SMM) and/or detecting early tumor and/or extramedullary MM disease.

In one aspect, the detecting step comprises detecting gadolinium. For example, the detecting step comprises detecting Gd¹⁵⁵ concentrations.

Also provided is a targeted nanoparticle conjugate comprising a nanoparticle comprising multiple sites of conjugation; and an anti-BCMA antibody.

### Definitions

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 0%, 0.5%, 0.1%, 0.05%, or 0.01% of the stated value. Unless otherwise clear from context, all numerical values provided herein are modified by the term “about.”

By “agent” is meant any small compound, antibody, nucleic acid molecule, or polypeptide, or fragments thereof.

The term “antibody” (Ab) as used herein includes monoclonal antibodies, polyclonal antibodies, multispecific antibodies (e.g., bispecific antibodies), and antibody fragments, so long as they exhibit the desired biological activity. The term “immunoglobulin” (Ig) is used interchangeably with “antibody” herein. The term “antibody” as used herein may refer to a variety of immunologically specific proteins. Although not within the term “antibody molecules,” the invention also includes “antibody analog(s),” other non-antibody molecule protein-based scaffolds, e.g., engineered binding proteins, fusion proteins and/or immunoconjugates that use CDRs to provide specific antigen binding. The term “antibody” also includes synthetic and genetically engineered variants.

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the antibody is purified: (1) to greater than 95% by weight of antibody as determined by the Lowry method, and most preferably more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The basic four-chain antibody unit is a heterotetrameric glycoprotein composed of two identical light (L) chains and two identical heavy (H) chains. An IgM antibody consists of 5 of the basic heterotetramer unit along with an additional polypeptide called J chain, and therefore contains 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has, at the N-terminus, a variable domain (V_{H}) followed by three constant domains (C_{H}) for each of the α and γ chains and four C_{H} domains for μ and ε isotypes. Each L chain has, at the N-terminus, a variable domain (V_{L}) followed by a constant domain (C_{L}) at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

The L chain from any vertebrate species can be assigned to one of two clearly distinct types, called kappa (κ) and lambda (λ), based on the amino acid sequences of their constant domains (C_{L}). Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated alpha (α), delta (δ), epsilon (ε), gamma (γ) and mu (μ), respectively. The γ and α classes are further divided into subclasses on the basis of relatively minor differences in C_{H} sequence and function, e.g., humans express the following subclasses: IgG1, IgG2, IgG3, IgG4, IgA1, and IgA2. The term “variable” refers to the fact that certain segments of the V domains differ extensively in sequence among antibodies. The V domain mediates antigen binding and defines specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. e al. Nucl. Acids Res. 28:219-221 (2000)). Optionally the antibody has symmetrical insertions at one or more of the following points 28, 36 (L1), 63, 74-75 (L2) and 123 (L3) in the V_{L}, and 28, 36 (H1), 63, 74-75 (H2) and 123 (H3) in the V_{H} when numbered in accordance with AHo; Honneger, A. and Plunkthun, A. J. Mol. Biol. 309:657-670 (2001)).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

Monoclonal antibodies include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81:6851-6855 (1984)). Also provided are variable domain antigen-binding sequences derived from human antibodies. Accordingly, chimeric antibodies of primary interest herein include antibodies having one or more human antigen binding sequences (e.g., CDRs) and containing one or more sequences derived from a non-human antibody, e.g., an FR or C region sequence. In addition, chimeric antibodies of primary interest herein include those comprising a human variable domain antigen binding sequence of one antibody class or subclass and another sequence, e.g., FR or C region sequence, derived from another antibody class or subclass. Chimeric antibodies of interest herein also include those containing variable domain antigen-binding sequences related to those described herein or derived from a different species, such as a non-human primate (e.g., Old World Monkey, Ape, etc). Chimeric antibodies also include primatized and humanized antibodies. Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992).

A “humanized antibody” is generally considered to be a human antibody that has one or more amino acid residues introduced into it from a source that is non-human. These non-human amino acid residues are often referred to as “import” residues, which are typically taken from an “import” variable domain. Humanization is traditionally performed following the method of Winter and co-workers (Jones et al., Nature, 321:522-525 (1986); Reichmann et al., Nature, 332:323-327 (1988); Verhoeyen et al., Science, 239:1534-1536 (1988)), by substituting import hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species.

A “human antibody” is an antibody containing only sequences present in an antibody naturally produced by a human. However, as used herein, human antibodies may comprise residues or modifications not found in a naturally occurring human antibody, including those modifications and variant sequences described herein. These are typically made to further refine or enhance antibody performance.

The phrase “functional fragment or analog” of an antibody is a compound having qualitative biological activity in common with a full-length antibody. For example, a functional fragment or analog of an anti-IgE antibody is one that can bind to an IgE immunoglobulin in such a manner so as to prevent or substantially reduce the ability of such molecule from having the ability to bind to the high affinity receptor, Fc_{ε}RI.

The term “antibody fragment” denotes a molecule other than an intact antibody that comprises a portion of an intact antibody that binds the antigen (e.g., BCMA) to which the intact antibody binds. Examples of antibody fragments include but are not limited to Fv, Fab, Fab′, Fab′-SH, F(ab′)₂, diabodies, linear antibodies, single-chain antibody molecules (e.g., scFv), and multispecific antibodies formed from antibody fragments.

The antibody(ies) used in the present method may be detected via detection of antibody-attached moieties (e.g., fluor and/or dye labeling, e.g., Cy5) or immunologically. That is, the presence of an antibody in the sample by be detected by an anti-antibody, such as an anti-IgG antibody labeled as may be found in indirect ELISAs, e.g. horseradish peroxidase (HRP) and alkaline phosphatase (AP). Other enzymes may be used as well. These include β-galactosidase, acetylcholinesterase and catalase. A large selection of substrates is available for performing the ELISA with an HRP or AP conjugate. The choice of substrate depends upon the required assay sensitivity and the instrumentation available for signal-detection (spectrophotometer, fluorometer or luminometer).

In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The term “administration” refers to introducing a substance into a subject. In general, any route of administration may be utilized including, for example, parenteral (e.g., intravenous), oral, topical, subcutaneous, peritoneal, intraarterial, inhalation, vaginal, rectal, nasal, introduction into the cerebrospinal fluid, or instillation into body compartments. In some embodiments, administration is oral. Additionally or alternatively, in some embodiments, administration is parenteral. In some embodiments, administration is intravenous.

By “control” or “reference” is meant a standard of comparison. In one aspect, as used herein, “changed as compared to a control” sample or subject is understood as having a level that is statistically different from a sample from a normal, untreated, or control sample. Control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., an antibody, a protein) or a substance produced by a reporter construct (e.g, β-galactosidase or luciferase). Depending on the method used for detection, the amount and measurement of the change can vary. Determination of statistical significance is within the ability of those skilled in the art, e.g., the number of standard deviations from the mean that constitute a positive result.

“Detect” refers to identifying the presence, absence, or amount of the agent (e.g., a nucleic acid molecule, for example deoxyribonucleic acid (DNA) or ribonucleic acid (RNA)) to be detected.

A “detection step” may use any of a variety of known methods to detect the presence of nucleic acid (e.g., methylated DNA) or polypeptide. The types of detection methods in which probes can be used include Western blots, Southern blots, dot or slot blots, and Northern blots.

As used herein, the term “diagnosing” refers to classifying pathology or a symptom, determining a severity of the pathology (e.g., grade or stage), monitoring pathology progression, forecasting an outcome of pathology, and/or determining prospects of recovery.

By “fragment” is meant a portion, e.g., a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. For example, a fragment may contain 10, 20, 30, 40, 50, 60, 70, 80, 90, or 100, 200, 300, 400, 500, 600, 700, 800, 900, or 1000 nucleotides or amino acids. However, the invention also comprises polypeptides and nucleic acid fragments, so long as they exhibit the desired biological activity of the full length polypeptides and nucleic acid, respectively. A nucleic acid fragment of almost any length is employed. For example, illustrative polynucleotide segments with total lengths of about 10,000, about 5000, about 3000, about 2,000, about 1,000, about 500, about 200, about 100, or about 50 base pairs in length (including all intermediate lengths) are included in many implementations of this invention. Similarly, a polypeptide fragment of almost any length is employed. For example, illustrative polypeptide segments with total lengths of about 10,000, about 5,000, about 3,000, about 2,000, about 1,000, about 5,000, about 1,000, about 500, about 200, about 100, or about 50 amino acids in length (including all intermediate lengths) are included in many implementations of this invention.

The term “in vitro” as used herein refers to events that occur in an artificial environment, e.g., in a test tube or reaction vessel, in cell culture, etc., rather than within a multi-cellular organism.

As used herein “in vivo” refers to events that occur within a multi-cellular organism, such as a human and a non-human animal. In the context of cell-based systems, the term may be used to refer to events that occur within a living cell (as opposed to, for example, in vitro systems).

The term “imaging agent” as used herein refers to any element, molecule, functional group, compound, fragments thereof or moiety that facilitates detection of an agent (e.g., a polysaccharide nanoparticle) to which it is joined. Examples of imaging agents include, but are not limited to: gadolinium, e.g., Gd¹⁵⁵, various ligands, radionuclides (e.g., 3H, 14C, 18F, 19F, 32P, 35S, 135I, 125I, 123I, 64Cu, 187Re, mIn, 90Y, 99mTc, 177Lu, 89Zr etc.), fluorescent dyes, chemiluminescent agents (such as, for example, acridinum esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes (for specific examples of enzymes, see below), colorimetric labels (such as, for example, dyes, colloidal gold, and the like), biotin, dioxigenin, haptens, and proteins for which antisera or monoclonal antibodies are available.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation.

By “marker” is meant any protein or polynucleotide having an alteration in expression level or activity that is associated with a disease or disorder.

As used herein, the term “nanoparticle” refers to a particle having a diameter of less than 1000 nanometers (nm). In some embodiments, a nanoparticle has a diameter of less than 300 nm, as defined by the National Science Foundation. In some embodiments, a nanoparticle has a diameter of less than 100 nm as defined by the National Institutes of Health. Optionally, a nanoparticle has a diameter of less than 50 nm, optionally less than 25 nm, optionally less than 20 nm, optionally less than 15 nm, optionally less than 10 nm, and optionally approximately 5 nm or less. In some embodiments, nanoparticles are micelles in that they comprise an enclosed compartment, separated from the bulk solution by a micellar membrane, typically comprised of amphiphilic entities which surround and enclose a space or compartment (e.g., to define a lumen). In some embodiments, a micellar membrane is comprised of at least one polymer, such as for example a biocompatible and/or biodegradable polymer.

As used herein, the term “subject” includes humans and mammals (e.g., mice, rats, pigs, cats, dogs, and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

Unless specifically stated or obvious from context, as used herein, the term “or” is understood to be inclusive. Unless specifically stated or obvious from context, as used herein, the terms “a”, “an”, and “the” are understood to be singular or plural.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, the term “treatment” (also “treat” or “treating”) refers to any administration of a substance that partially or completely alleviates, ameliorates, relives, inhibits, delays onset of, reduces severity of, and/or reduces incidence of one or more symptoms, features, and/or causes of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIG. 1A to FIG. 1J depict the process employed and results obtained that guided rational selection and design of a useful targeted imaging contrast agent (biomarker) for MM. FIG. 1A shows a volcano plot that compares the expression level of BCMA and signaling lymphocytic activation molecule F7 (SLAMF7) as a function of the disease stage (MGUS, SMM, MM and relapsed, respectively) of patients from the Achilles dataset that was analyzed. FIG. 1B shows a schematic representation of conjugation via a homobifunctional linker (represented in green) using NHS chemistry of a gadolinium-based silica nanoparticles (Gd—NPs) to monoclonal antibodies targeting malignant plasma cells (e.g., cells expressing BCMA as a cell-surface biomarker). FIG. 1C shows hydrodynamic sizes observed for the nanoparticle (NP), in non-conjugated form and as nanoparticle-antibody complexes of Gd—NPs (NP) with anti-SLAMF7 (NP-SLAMF7) and anti-BCMA antibodies (NP-BCMA), respectively (traces from left to right). FIG. 1D shows corresponding observed relaxivity (r1) values for each NP-containing composition, as assessed using a 7T MRI machine. FIG. 1E shows competitive labeling of MM1.S cells with Cy5.5-conjugated anti-BCMA antibodies and either Gd—NPs (NP) or NP-BCMA as assessed by flow cytometry, which demonstrated that inclusion of anti-BCMA antibody as a NP conjugate promoted the binding of SiGdNP to MM1.S cells. FIG. 1F shows fluorescent confocal imaging that confirmed the colocalization of anti-BCMA antibodies (AF488 signal) and Gd—NPs (Cy5-bound signal) on the surfaces of DAPI-stained plasma cells administered the NP-anti-BCMA conjugate, therefore confirming the effective targeting of this conjugate composition (that included anti-BCMA conjugated with the nanoparticles) to the plasma cell nucleus. Bar scale=5 μm. FIG. 1G shows imaging of GFP⁺/Luc⁺ MM1.S cells (arrowheads) in mice by MRI at 19 days after implantation and after the administration of various contrast agents (n=5 mice/group)—images are specifically for mice that were initially intravenously injected with MM1.S_{GFP}⁺_{/Luc}⁺ cells which were allowed to disseminate for 19 days. Afterward, n=5/group were imaged with Magnevist, NP, or NP conjugated to a monoclonal antibody (anti-SLAMF7 or anti-BCMA), respectively. Arrowheads indicate targeting of NP-monoclonal antibody conjugates to the spine in such mice. FIG. 1H shows hematoxylin and eosin (H&E) staining, which was used to confirm the presence of plasma cells in the bone marrow, and Prussian staining, which showed the presence of gadolinium (Gd; highlighted with arrows). Scale bar=50 nm. FIG. 1I shows normalized signal-to-noise ratios (SNR) observed for the spine of treated mice over time, as normalized to baseline acquisition levels. FIG. 1J shows the result of a biodistribution study of the NP-BCMA in non-tumor-bearing mice, as assessed by quantification of the gadolinium concentration over time (percentages of the injected Gd dose per gram (% ID/g) in various organs) by ICP-MS (n=5/time point). The sub-image of FIG. 1J represents the amount of gadolinium (Gd) observed (from the free NP) in spines and femurs of each healthy animal. * P<0.05, ** P<0.005, *** P<0.001.

FIG. 2A to FIG. 2L show validation of the anti-BCMA targeting imaging biomarker (NP-anti-BCMA monoclonal antibody conjugate) for MRD detection, with MRI of the NP-BCMA conjugate demonstrating its utility as such a novel biomarker. In FIG. 2A to FIG. 2C, animals were injected intravenously with MM1.S_{GFP}^{+/}_{LUC}⁺ and imaged once a week by bioluminescence imaging (FIG. 2A), MRI at 30 min after an injection of NP-BCMA (FIG. 2B) or CT scans (FIG. 2C) to visualize tumor burden (arrows). After 21 days (day 21 after tumor cell implantation), a model for minimal residual disease (MRD) was induced by administering a treatment of Bortezomib (3×0.5 mg/kg) and Melphalan (5.5 mg/kg), with the MRD model established at day 25. Afterward, mice continued to be imaged once a week to follow their disease burdens (the MRD status). FIG. 2D shows the change of BLI signal intensities observed. FIG. 2E shows the MRI signal-to-noise ratio changes observed. FIG. 2F shows the result of CT quantification and assessment for tumor presence, with changes in CT SNR specifically quantified to assess the detection of tumor cells. FIG. 2G shows results obtained when lambda light-chain levels were quantified by immunoassay. Shadowing demarcates the 90% confidence interval (n=5 per group). FIG. 2H shows the receiving operator characteristic (ROC) curve observed at week 5, comparing the sensitivity and specificity of the 4 modalities to detect the presence of MRD. Dashed line represents a diagnostic modality with no discriminatory power (AUC=0.50). FIG. 2I shows a comparison of the area under the curve (AUC) observed over the course of the treatment, specifically comparing the sensitivity and specificity of the 4 detection modalities. FIG. 2J shows flow cytometry histograms depicting the percentages of total plasma cells (GFP signal) and NP-BCMA-bound plasma cells (Cy5.5 signal) at each time point. FIG. 2K shows the total percentages of plasma cells, as enumerated and compared (n=3 mice per group) at the indicated time points. FIG. 2L shows the percentages of NP-BCMA-bound plasma cells in the bone marrow, as enumerated and compared (n=3 mice per group) at the indicated time points.

FIG. 3A to FIG. 3D show conjugation of the gadolinium-based nanoparticles to monoclonal antibodies. FIG. 3A shows HPLC measurements that confirmed the presence of anti-BCMA antibodies before (left—free NP) and after conjugation to the gadolinium-based nanoparticles (Gd—NPs, right—NP-BCMA) in purified suspensions. FIG. 3B shows a PACE experiment that confirmed binding of the anti-BCMA antibodies to the Gd—NPs. FIG. 3C and FIG. 3D show DLS measurements that demonstrated a stable nanoparticle size post-conjugation over time and in acidic pH condition—in particular, the stability of various nanoparticle suspensions before (Gd—NP) and after conjugation to either anti-BCMA antibodies (FIG. 3C) or anti-SLAMF7 (FIG. 3D) over time and in acidic pH conditions was confirmed.

FIG. 4A and FIG. 4B show in vitro binding efficiency of various NP-antibody complexes (including the NP-anti-BCMA conjugates and the NP-anti-SLAMF7 conjugates) to malignant plasma cells. FIG. 4A shows FACS data showing that the NP-anti-BCMA conjugate targeted BCMA antigens on MM1.S cells—specifically, percentages of fluorescently labeled MM1.S cells as determined by flow cytometry of fluorescently-labeled nanoparticles alone (NP) or after their further conjugation to anti-BCMA antibodies (NP-BCMA) were determined. FIG. 4B shows a gadolinium uptake study by ICP-MS after 30 min of incubation—specifically, gadolinium (Gd) uptake by various MM cell lines was assessed, as determined by ICP-MS of cell lysates performed after 30 min of incubation with unmodified (NP), anti-SLAMF7 antibody-conjugated nanparticles (NP-SLAMF7), or anti-BCMA antibody-conjugated nanoparticles (NP-BCMA).

FIG. 5A and FIG. 5B show a cell survival assay that demonstrated the non-toxicity of the nanoparticles of the instant disclosure. Relative in vitro toxicity of nanoparticle-antibody complexes was determined via assessment of cellular viabilities of different MM cell lines which were examined by CellTiter 96 Aqueous One Solution Proliferation Assay as a function of incubation with increasing concentrations of monoclonal antibodies alone (anti-SLAMF7, anti-BCMA), gadolinium-based nanoparticles (Gd—NPs) alone, or nanoparticle-antibody complexes (NP-SLAMF7 or NP-BCMA), FIG. 5A specifically shows a toxicity evaluation of the two monoclonal antibodies alone and the gadolinium nanoparticles alone. FIG. 5B shows a toxicity evaluation of the nanoparticle-antibody complexes (NP-BCMA and NP-SLAMF7 nanoparticle conjugates). All experiments were performed at 72 h post-incubation with nanoparticles.

FIG. 6 demonstrates MM1.S tumor dissemination by bioluminescence imaging (BLI), specifically showing the growth of plasmacytomas in an orthotopic cell-line xenograft model of multiple myeloma. Human GFP⁺/LUC⁺ MM1.S cells were introduced into 4 mice via IV dissemination and BLI was performed at various days thereafter. For the MRI studies, the models were similarly established and mice were included in the study at day 19 after tumor cell implantation, which was the time point at which the tumor burden of their femurs and spines could be readily discerned.

FIG. 7 shows nanoparticle uptake in the femur sites of MM1.S-bearing mice at day 19 post tumor cell implantation. At left, five mice were imaged after IV administration of either NP-SLAMF7 (top) or NP-BCMA (bottom). At right, the contrast to noise (CNR) ratio in their femurs was determined at various time points after injection of the nanoparticle-antibody complexes. * p-values<0.05, *** p-values<0.001, two-tailed t-test.

FIG. 8A and FIG. 8B show a histological assessment of tumor burden by H&E (left) and the locations of nanoparticle-antibody complexes (nanoparticle uptake) as determined by Prussian blue staining (right) in the spine (FIG. 8A) and the femurs (FIG. 8B) of mice injected with NP-anti-BCMA conjugates.

FIG. 9A to 9F show biodistribution, pharmacokinetic and toxicity evaluation of the different nanoparticles (gadolinum-based nanoparticles and their antibody complexes). FIG. 9A shows biodistribution (tissue distribution) of the NP (unconjugated (NP), anti-SLAMF7 antibody-conjugated (NP-SLAMF7) and anti-BCMA antibody-conjugated Gd-based nanoparticles (NP-BCMA)) in non-tumor-bearing (healthy) mice; quantifications of the percentages of the injected Gd dose per gram (% ID/g) in various organs were measured by ICM-MS over time (n=5/time point). FIG. 9B shows a pharmacokinetic study performed upon serial blood samples drawn from the same animal, measured by ICM-MS (n=5/time points), which showed changes in the Gd-concentrations in blood from healthy mice administered NP, NP-SLAMF7, or NP-BCMA. FIG. 9C shows the influence of the nanoparticle on the body weight variation that was observed over time (n=5 mice/time point)—changes in the body weights of healthy mice were observed as a function of time after administration of a single dose of various Gd-based contrast agents. FIG. 9D shows basic metabolic profiles (n=2 mice per group), while FIG. 9E shows complete blood counts and FIG. 9F shows a chemical panel comparison (white blood cell differential counts) from healthy mice between a control group (n=3 mice) and an experimental group at 96 h after injection of the NP-anti-BCMA conjugate (n=3 mice).

FIG. 10 shows H&E staining of organ from healthy mice that were sacrificed at various time points after administration of a single dose of NP-BCMA, which was used to assess the toxicity of the NP-anti-BCMA conjugate over time. No toxicity was observed from the H&E slides, which confirmed the safety profile of the NP-anti-BCMA conjugate.

### DETAILED DESCRIPTION OF THE INVENTION

The present disclosure is directed, at least in part, to nanoparticle-antibody conjugates targeted to cell surface receptors—conjugates which, because of their targeted nature, possess enhanced ability as imaging agents for detection and localization of multiple myeloma and/or the presence of MRD in a cell line and/or subject. In certain embodiments, the nanoparticle moieties of the antibody-nanoparticle conjugates of the instant disclosure are gadolinium-based and optionally are of such small size (e.g., NPs of less than 5 nm) that such conjugate compositions, even when conjugated to targeting moieties (e.g., anti-BCMA monoclonal antibodies) via linkers (e.g., NHS linker moieties), are relatively rapidly cleared from the circulation of a subject via renal excretion, with no toxic impact. Thus, the nanoparticle-antibody conjugates described herein provide improved imaging contrast and allow for enhanced monitoring of MRD and/or therapeutic prediction of MM.

Many new therapeutic modalities for MM are currently in clinical trials, and recent rapid development of such compositions has been accompanied by an increased need for specific imaging biomarkers to monitor MRD, with the goal of improving the evaluation and efficacy of such treatments. Most MM patients are diagnosed as MRD positive, due to a rapid variation of M-spike/free light-chain (FLC) ratio level and/or end organ damage, as indicated by, e.g., elevated calcium rate, renal failure, anemia, and/or bone lesion (CRAB criteria; Kumar et al. Lancet Oncol 17: e328-346). Where the M-spike/FLC level ratio increases, patients are imaged by whole body-X ray to detect bone lesions. However, this imaging technique lacks sensitivity. Because new MM therapies have become available, early confirmation of MM can significantly improve patient outcomes.

Minimal residual disease (MRD) is directly linked to both shorter durations of treatment response as well as to inferior long-term survival outcomes in patients with multiple myeloma (MM; Kumar et al. Lancet Oncol 17: e328-346; Nishihori et al. Curr Hematol Malig Rep 11: 118-126; Anderson et al. Clin Cancer Res, doi:10.1158/1078-0432.CCR-16-2895). Current diagnostic methods that utilize serologic studies and/or bone marrow examinations do not take into account the spatial heterogeneity of the tumor microenvironment; they require serial invasive samplings to diagnose residual plasma cells. Available diagnostic imaging modalities are not sensitive nor specific for the detection of malignant plasma-cells (Lapa et al. Theranostics 6: 254-261) and often rely on ionizing radiation that precludes frequent testing (Fazel et al. N Engl J Med 361: 849-857).

It is predicted that establishment of imaging methods for the detection of MRD will have a transformative impact on the care of patients with MM, enabling noninvasive and repetitive testing to find residual plasma cells at earlier time points and when present even in focal distribution patterns that would otherwise preclude detection.

Magnetic resonance imaging (MRI) is known to provide a more reliable method for assessing disease burden, prognosis, and to monitor response to therapy, as compared to computed tomography (CT) scans and positron emission tomography (PET) (Spinnato P. et al, Eur J Radiol. 2012 81(12):4013-8). Techniques for magnetic resonance imaging (MRI) with conventional FDA-approved agents are being developed and have been shown to be more reliable at assessing disease burden (Pawlyn et al. Leukemia 30: 1446-1448), for enabling accurate disease prognostication (Dimopoulos et al. J Clin Oncol 33: 657-664), as well as for following therapeutic responses in MM patients when compared to computed tomography (CT), single-photon emission computed tomography (SPECT), or positron emission tomography (PET; Spinnato et al. Eur J Radiol 81: 4013-4018). MRI has the advantage of distinguishing between benign and malignant osteolytic regions, in addition to detecting early marrow infiltration (Shortt et al. AJR Am J Roentgenol 192: 980-986); however, the current protocols used to perform MRI—i.e., fat-water imaging, diffusion weighted imaging, contrast enhancement—are time-consuming, expensive, and rely on passive accumulation of non-targeted constrast agents within the tumor microenvironment (Matsumura and Maeda. Cancer Res 46: 6387-6392), which has hitherto limited both their detection specificity and sensitivity. CT scans can only detect bone destruction but not myeloma activity, and PET imaging is reliant upon imaged cells exhibiting active metabolism yet PET imaging does not possess sufficient sensitivity to visualize residual MM cells displaying slow proliferative activity (Freedenberg M I et al. Phys Med 2014 30(1):104-10). While SPECT and fluorodeoxyglucose-based (¹⁸F-FDG-) PET are able to accurately identify plasma cell populations (Cavo et al. Lancet Oncol 18: e206-e217), they utilize ionizing radiation that prevents repetitive testing in short intervals. ¹⁸F-FDG-PET also displays poor detection sensitivity for malignant plasma cells in the MRD state, which are more slowly proliferative.

Prior to the invention described herein, there was a pressing need for an imaging biomarker for MM based on MRI acquisition, which would present clear advantages over existing imaging techniques. In MM, and more specifically for MRD diagnostics, the current MRI contrast agents (gadolinium chelates) rely upon the passive targeting pathway (Zhou et al. Wiley Interdiscip Rev Nanomed Nanobiotechnol. 2013 5(1):1-18) (EPR-effect), which does not allow for production of a contrast signal that is sufficiently specific to be detected. In nanomedicine, use of antibodies has been proposed for targeting of cell-surface receptors. However, recent proof-of-concept studies performed upon such targeting agents have demonstrated suboptimal results. Indeed, NP circulation half-time time was observed to have dropped dramatically in such studies, resulting in a low in vivo binding affinity due to the large size of these complexes, and imaging was further prevented by the inability of the NP to escape the vasculature in order to target the cell surface receptors to which the antibody was targeted. As a result, no difference was observed between passive and actively targeted forms of NP, limiting their application as effective and specific imaging agents. As described in detail below, to develop an improved imaging biomarker and noting that a primary issue to address was the final size of the complex “nanoparticle-full antibody”, an ultrafine sub-5 nm NP having high MRI properties was selected, as were relatively small monoclonal antibodies. The instant disclosure focuses upon generating a novel MM-targeted contrast agent capable of using short MRI sequences to identify minute tumor cell populations with high spatial localization. A primary goal of the current disclosure was to generate gadolinium (Gd)-based nanoparticles (Gd—NPs) that could be specifically targeted to plasma cells to enhance early detection of MRD. The use of antibodies has long been proposed for targeting of nanoparticles to tumors by binding receptors that are overexpressed on their cell surfaces (Ulbrich et al. Chem Rev 116: 5338-5431; Mulvey et al. Nat Nanotechnol 8: 763-771). As the sizes of these typical nanoparticle-antibody complexes (50-200 nm in diameter; Arruebo et al. J Nanomater, doi:10.1155/2009/439389 (2009)) are much larger than those of full monoclonal antibodies or of their molecular-conjugates (10-15 nm in length and 3-5 nm in diameter; Reth, M. Nat Immunol 14: 765-767), their pharmacology has been largely dictated by the nanoparticle rather than by the antibody. Moreover, most preclinical studies performed with such agents have been conducted in subcutaneous xenograft models (Smith et al. Nat Nanotechnol, doi:10.1038/nnano.2017.57 (2017); Qian et al. Nat Biotechnol 26: 83-90) that do not recapitulate the vascular patterns found in the natural tumor microenvironment (Mack and Marshall. Nat Biotechnol 28: 214-229). Many of these reported constructs have, thus, exhibited no differences with respect to their untargeted counterparts in achieving tumor localization (Kunjachan et al. Nano Lett 14: 972-981), which have stymied their further translational development.

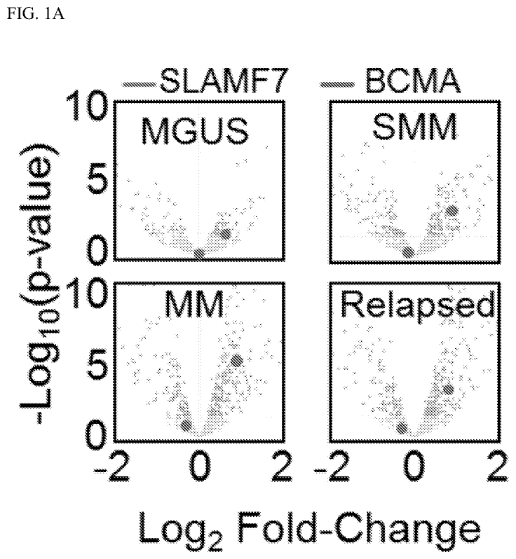

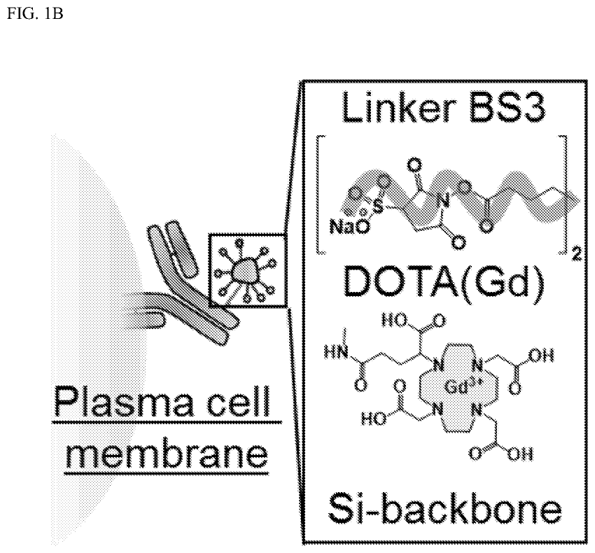

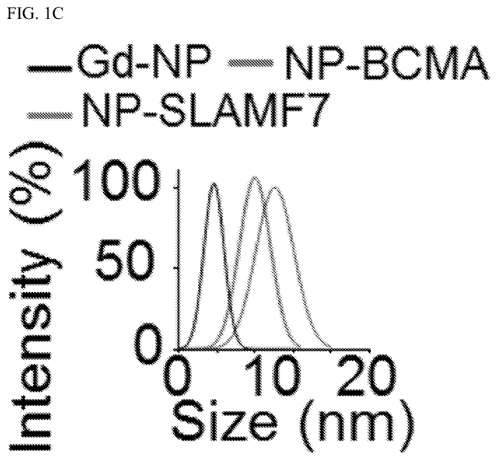

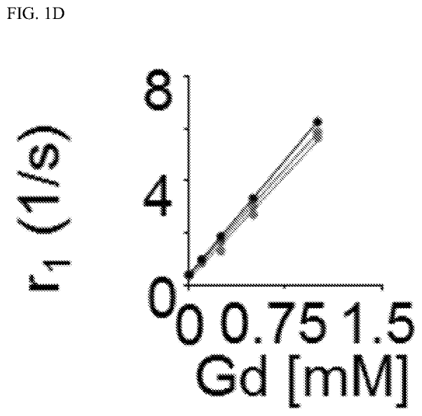

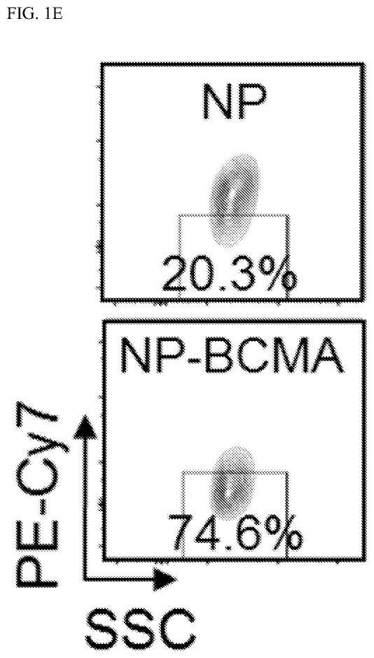

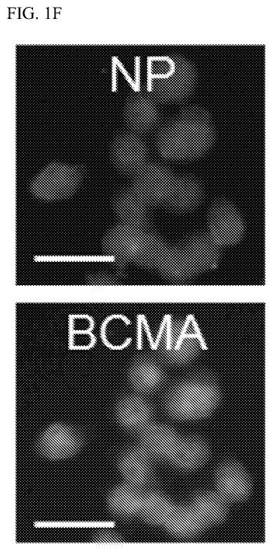

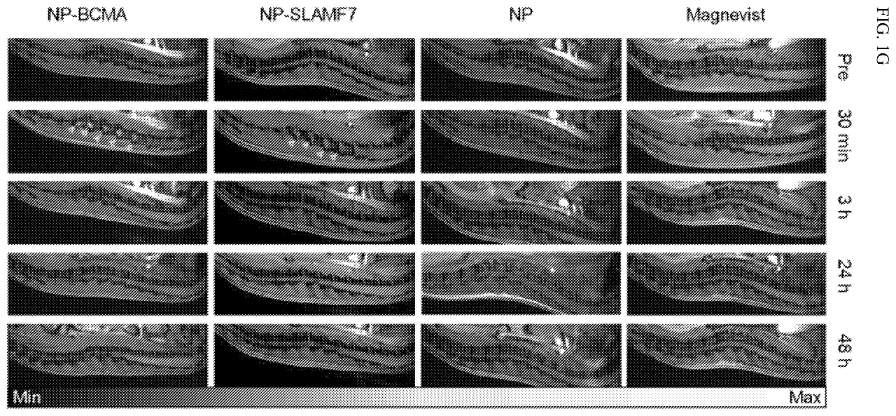

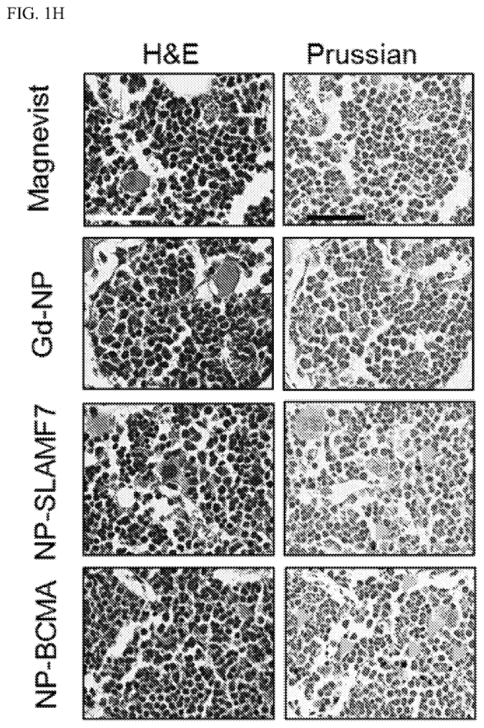

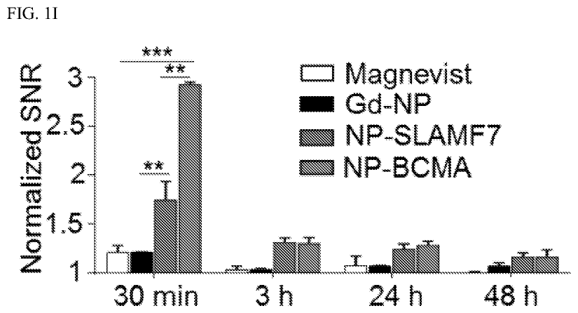

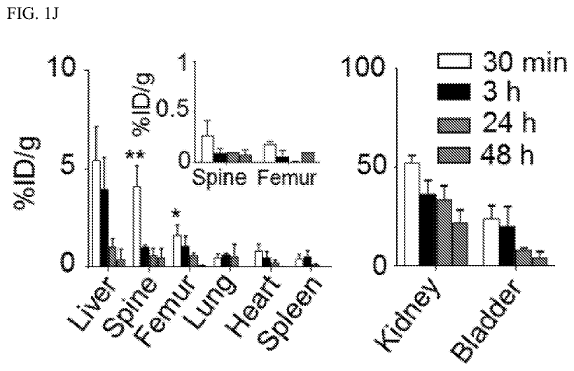

The targeting efficiencies of monoclonal antibodies directed to two specific antigens—the B-cell maturation antigen (BCMA) and the signaling lymphocytic activation molecule-F7 (SLAMF7) receptor—were initially compared. Both targets are well-established antigens almost exclusively present on the cell surface of non-malignant B-cells (Lonial et al. N Engl J Med 373:621-631; Novak et al. Blood 103:689-694). BCMA, as distinguished from SLAMF7, is a highly specific plasma cell antigen having an important role in the maturation and differentiation of the B-cell into a plasma cell (Carpenter R O et al. Clin Cancer Res 201319(18):2048-60). The high prevalence and expression level of BCMA increases with the advancement of the MM progression (FIG. 1A), rendering BCMA an ideal cell-surface receptor for monitoring of MM. Described herein is the development of a conjugate of a sub-5 nm NP (as described in Detappe et al. Nano Lett 17:1733-1740; Detappe et al. J Control Release 238:103-113), which is a silica-based gadolinium NP, that is specifically targeted to the cell-surface receptors of plasma cells, and which thereby allows for more efficient and specific prediction (enhanced specificity and sensitivity) of MM disease progression and/or the outcomes of MM therapies, including newly-developed MM therapies.

Herein, magnetic resonance imaging (MRI) of ultra-small gadolinium-based nanoparticles that were conjugated to monoclonal antibodies has been utilized to enable rapid detection of clonal plasma cells in the bone marrow microenvironment. It is believed that the instant disclosure represents the first example of utilizing a non-invasive and safe imaging agent to improve early detection of MRD after therapeutic administration.

Certain targeted nanoparticle conjugates of the disclosure are capable of enhancing the sensitivity of detecting MM cells in a subject (e.g., in a mammalian subject). Targeted nanoparticles of the disclosure can, for example, improve sensitivity by at least 1.5-fold relative to untargeted NPs. Optionally, sensitivity is improved by at least two-fold relative to untargeted NPs. Optionally, sensitivity is improved by at least three-fold relative to untargeted NPs. Optionally, sensitivity is improved by at least five-fold relative to untargeted NPs. Optionally, sensitivity is improved by at least ten-fold relative to untargeted NPs.

Certain targeted nanoparticle conjugates of the disclosure can additionally and/or alternatively enhance the specificity of detecting MM cells in a subject (e.g., in a mammalian subject). Targeted nanoparticles of the disclosure can, for example, improve specificity by at least 1.5-fold relative to untargeted NPs. Optionally, specificity is improved by at least two-fold relative to untargeted NPs. Optionally, specificity is improved by at least three-fold relative to untargeted NPs. Optionally, specificity is improved by at least five-fold relative to untargeted NPs. Optionally, specificity is improved by at least ten-fold relative to untargeted NPs.

In certain embodiments, a targeted nanoparticle conjugate of the disclosure can possess a lower MRI detection threshold for MRD than a non-targeted nanoparticle. For example, the MRI detection threshold for MRD in a subject for certain targeted nanoparticles of the disclosure can be 100,000 or less plasma cells per subject, optionally 50,000 or less plasma cells per subject, optionally 30,000 or less plasma cells per subject, optionally 20,000 or less plasma cells per subject, optionally 10,000 or less plasma cells per subject, optionally 8,000 or less plasma cells per subject, optionally 6,000 or less plasma cells per subject, optionally 5,000 or less plasma cells per subject, optionally 4,000 or less plasma cells per subject, optionally 3,000 or less plasma cells per subject, optionally about 2,200 plasma cells per subject—e.g., optionally 2,200±450 plasma cells per subject (optionally, where the subject is a mouse).

### Anti-BCMA Monoclonal Antibodies

B cell maturation antigen (BCMA) is member 17 of the tumor necrosis factor receptor superfamily (TNFRSF). Its native ligands are the B cell activating factor (BAFF; also called BLγS or TALL-1, TNFSF13B) and a proliferation-inducing ligand (APRIL, TNFSF13, CD256) (Mackay et al. (2003) Annu Rev Immunol 21:231-264) which are ultimately involved (through interaction with further ligands) in regulating various aspects of humoral immunity, B cell development, and homeostasis. The affinity for BAFF lies in the low micromolar range whereas APRIL binds nearly 100 fold tighter to BCMA (Bossen et al. (2006) Semin Immunol 18:263-275). Expression of BCMA is restricted to the B cell lineage where it is predominantly expressed on plasma blasts and plasma cells but is absent from naive B cells, germinal center B cells and memory B cells (Darce et al. (2007) J Immunol 179:7276-7286; Benson et al. (2008) J Immunol 180:3655-3659; Good et al. (2009) J Immunol 182:890-901).

BCMA expression is important for the survival of long-lived, sessile plasma cells in the bone marrow (O'Connor et al. (2004) J Exp Med 199:91-98). Consequently, BCMA-deficient mice show reduced plasma cell numbers in the bone marrow whereas the level of plasma cells in the spleen in unaffected (Peperzak et al. (2013) Nat Immunol [Epub 2013 Feb. 3, 10.1038/ni.2527]). The differentiation of mature B cells into plasma cells is normal in BCMA knockout mice (Schiemann et al. (2001) Science 293:21 1 1-21 14; Xu et al. (2001) Mol Cell Biol 21:4067-4074). The binding of BAFF or APRIL to BCMA triggers NF-κB activation (Hatzoglou et al. (2000) J Immunol 165:1322-1330), which induces upregulation of anti-apoptotic Bcl-2 members such as Bcl-xL or Bcl-2 and Mcl-1 (Peperzak et al. (2013) Nat Immunol [Epub 2013 Feb. 3, 10.1038/ni.2527]).

BCMA is also highly expressed on malignant plasma cells, for example in multiple myeloma, (MM), which is a B cell non-Hodgkin lymphoma of the bone marrow, and plasma cell leukemia (PCL), which is more aggressive than MM and constitutes around 4% of all cases of plasma cell disorders. In addition to MM and PCL, BCMA has also been detected on Hodgkin and Reed-Sternberg cells in patients suffering from Hodgkin's lymphoma (Chiu et al. (2007) Blood 109:729-739). Similar to its function on plasma cells, ligand binding to BCMA has been shown to modulate the growth and survival of multiple myeloma cells expressing BCMA (Novak et al. (2004) Blood 103:689-694). Signaling of BAFF and APRIL via BCMA are considered as pro-survival factors for malignant plasma cells; hence, the depletion of BCMA-positive tumor cells and/or the disruption of ligand-receptor interaction should improve the therapeutic outcome for multiple myeloma and autoantibody-dependent autoimmune diseases. There are presently various approaches available for the treatment of multiple myeloma (Raab et al. (2009) Lancet 374:324-339). Chemotherapy leads in most subjects only to partial control of multiple myeloma; only rarely does chemotherapy lead to complete remission. Combination approaches are therefore often applied, commonly involving an additional administration of corticosteroids, such as dexamethasone or prednisone. Corticosteroids are, however, plagued by side effects, such as reduced bone density. Stem cell transplantation has also been proposed, using one's own stem cells (autologous) or using cells from a close relative or matched unrelated donor (allogeneic). In multiple myeloma, most transplants performed are of the autologous kind. Such transplants, although not curative, have been shown to prolong life in selected patients (Suzuki (2013) Jpn J Clin Oncol 43:1 16-124). Alternatively, thalidomide and derivatives thereof have recently been applied in treatment but are also associated with sub-optimal success rates and high costs. More recently, the proteasome inhibitor bortezomib (PS-341) has been approved for the treatment of relapsed and refractory MM and was used in numerous clinical trials alone or in combination with established drugs resulting in an encouraging clinical outcome (Richardson et al. (2003) New Engl J Med 348:2609-2617; Kapoor et al. (2012) Semin Hematol 49:228-242). Therapeutic approaches are often combined. The costs for such combined treatments are correspondingly high and success rates still leave significant room for improvement. The combination of treatment options is also not ideal due to an accumulation of side effects if multiple medicaments are used simultaneously.

The ability to specifically target plasma cells is also of great benefit for the treatment of autoimmune diseases. Conventional therapy for autoimmune conditions such as systemic lupus erythematosus (SLE) and rheumatic arthritis (RA), in which autoreactive antibodies are crucial to disease pathology, depend on the severity of the symptoms and the circumstances of the patient (Scott et al. (2010) Lancet 376:1094-1 108, D'Cruz et al. (2007) Lancet 369, 587-596). In general, mild forms of disease are first treated with nonsteroidal antiinflammatory drugs (NSAID) or disease-modifying anti-rheumatic drugs (DMARD). More severe forms of SLE, involving organ dysfunction due to active disease, usually are treated with steroids in conjunction with strong immunosuppressive agents such as cyclophosphamide, a cytotoxic agent that targets cycling cells. Only recently Belimumab, an antibody targeting the cytokine BAFF, which is found at elevated levels in serum of patients with autoimmune diseases, received approval by the Food and Drug Administration (FDA) for its use in SLE. However, only newly formed B cells rely on BAFF for survival in humans, whereas memory B cells and plasma cells are less susceptible to selective BAFF inhibition (Jacobi et al. (2010) Arthritis Rheum 62:201-210). For rheumatoid arthritis, TNF inhibitors were the first licensed biological agents, followed by abatacept, rituximab, and tocilizumab and others: they suppress key inflammatory pathways involved in joint inflammation and destruction, which, however, comes at the price of an elevated infection risk due to relative immunosuppression (Chan et al. (2010) Nat Rev Immunol 10:301-316, Keyser (201 1) Curr Rheumatol Rev 7:77-87). Despite the approval of these biologicals, patients suffering from RA and SLE often show a persistence of autoimmune markers, which is most likely related to the presence of long-lived, sessile plasma cells in bone marrow that resist e.g. CD20-mediated ablation by rituximab and high dosage glucocorticoid and cyclophosphamid therapy. Current strategies in SLE include a “reset” of the immune system by immunoablation and autologous stem cell transplantation, though the risk for transplant-related mortality remains a serious concern (Farge et al. (2010) Haematologica 95:284-292). The use of proteasome inhibitors such as Bortezomib might be an alternative strategy for plasma cell depletion: owing to the high rate of protein synthesis and the limited proteolytic capacity, plasma cells are hypersensitive to proteasome inhibitors. Bortezomib has recently been approved for the treatment of relapsed multiple myeloma and a recent study in mice with lupus-like disease showed that bortezomib depletes plasma cells and protects mice with lupus-like disease from nephritis (Neubert et al. (2008) Nat Med 14:748-755). However, proteasome inhibitors do not specifically act on plasma cells and the incidence of adverse effects such as peripheral neuropathy is high (Arastu-Kapur et al. (2011) Clin Cancer Res 17:2734-2743).

Therapeutic antibodies can act through several mechanisms upon binding to their target. The binding itself can trigger signal transduction, which can lead to programmed cell death (Chavez-Galan et al. (2009) Cell Mol Immunol 6:15-25). It can also block the interaction of a receptor with its ligand by either binding to the receptor or the ligand. This interruption can cause apoptosis if signals important for survival are affected (Chiu et al. (2007) Blood 109:729-739). With regard to cell-depletion there are two major effector mechanisms known. The first is the complement-dependent cytotoxicity (CDC) towards the target cell. There are three different pathways known. However, in the case of antibodies the important pathway for CDC is the classical pathway which is initiated through the binding of C1 q to the constant region of IgG or IgM (Wang and Weiner (2008) Expert Opin Biol Ther 8:759-768).

The second mechanism is called antibody-dependent cellular cytotoxicity (ADCC). This effector function is characterized by the recruitment of immune cells which express Fc-receptors for the respective isotype of the antibody. ADCC is largely mediated by activating Fc-gamma receptors (FcyR) which are able to bind to IgG molecules either alone or as immune complexes. Mice exhibit three (FcyRI, FcyRIII and FcyRIV) and humans five (FcyRI, FcyRIIA, FcyRIIC, FcyRIIIA and FcyRIIIB) activating Fcy-receptors. These receptors are expressed on innate immune cells like granulocytes, monocytes, macrophages, dendritic cells and natural killer cells and therefore link the innate with the adaptive immune system.

Depending on the cell type, there are several modes of action of FcgR-bearing cells upon recognition of an antibody-marked target cell. Granulocytes generally release vasoactive and cytotoxic substances or chemoattractants but are also capable of phagocytosis. Monocytes and macrophages respond with phagocytosis, oxidative burst, cytotoxicity, or the release of pro-inflammatory cytokines, whereas Natural killer cells release granzymes and perforin and can also trigger cell death through the interaction with FAS on the target cell and their Fas ligand (Nimmerjahn and Ravetch (2008) Nat Rev Immunol 8:34-47; Wang and Weiner (2008) Expert Opin Biol Ther 8:759-768; Chavez-Galan et al. (2009) Cell Mol Immunol 6:15-25).

Antibodies which bind CD269 (BCMA) and their use in the treatment of various B-cell related medical disorders are described in the art. Ryan et al (Molecular Cancer Therapeutics, 2007 6 (11), 3009) describe an anti-BCMA antibody obtained via vaccination in rats using a peptide of amino acids 5 to 54 of the BCMA protein. The antibody described therein binds BCMA, blocks APRIL-dependent NF-KB activation and induces ADCC. No details are provided on the specific epitope of the antibody. WO 2012/163805 describes BCMA binding proteins, such as chimeric and humanized antibodies, their use to block BAFF and/or APRIL interaction with BCMA, and their potential use in treating plasma cell malignancies such as multiple myeloma. The antibody disclosed therein was obtained via vaccination in mouse using a recombinant peptide of amino acids 4 to 53 of the BCMA protein. WO 2010/104949 also describes various antibodies that bind preferably the extracellular domain of BCMA and their use in treating B cell mediated medical conditions and disorders. No details are provided on the specific epitope of the antibodies.

WO 2002/066516 describes bivalent antibodies that bind both BCMA and TACI and their potential use in the treatment of autoimmune diseases and B cell cancers. An undefined extracellular domain of BCMA is used to generate the anti-BCMA portion of the antibodies described therein. WO 2012/066058 discloses bivalent antibodies that bind both BCMA and CD3 and their potential use in the treatment of B cell related medical disorders. Details regarding the binding properties and specific epitopes of the antibodies are not provided in either publication.

WO 2012/143498 describes methods for the stratification of multiple myeloma patients involving the use of anti-BCMA antibodies. Preferred antibodies are those known as “Vicky-1” (lgG1 subtype from GeneTex) and “Mab 193” (lgG2a subtype from R&D Systems). Details regarding the binding properties and specific epitopes of the antibodies are not provided.

WO 2014/068079 describes an anti-BCMA antibody evaluated as suitable for use in the treatment of plasma cell diseases such as multiple myeloma (MM) and autoimmune diseases. WO 2014/068079 provides an isolated antibody or antibody fragment that binds CD269 (BCMA), in particular an epitope of the extracellular domain of CD269 (BCMA). An isolated antibody or antibody fragment that binds CD269 (BCMA) was therefore provided, wherein the antibody binds an epitope comprising one or more amino acids of residues 13 to 32 of CD269 (BCMA).

To raise an anti-BCMA antibody, antigen comprising the extracellular domain of CD269 was used in vaccination in order to generate the binding specificity of the anti-BCMA antibody. Use of the entire CD269 protein, or fragments thereof comprising either a membrane-bound or intracellular domain, as an antigen during antibody generation could produce antibodies that bind concealed or intracellular domains of CD269, thereby rendering such agents unsuitable or disadvantageous for therapeutic application. The antibodies described in WO 2014/068079 were therefore defined by their binding to the extracellular portion of CD269. The specific epitope within the extracellular domain also represented a preferred novel and unexpected characterising feature of the WO 2014/068079 publication.

Fab fragments prepared from one embodiment of the WO 2014/068079 were crystallized in complex with the purified BCMA extracellular domain and the complex structure solved. The structural analysis revealed detailed information of the epitope of the anti-BCMA antibody of the WO 2014/068079 publication and its biological relevance. The binding of an epitope comprising one or more amino acids of residues 13 to 32 of CD269 (BCMA) of the extracellular domain by the antibody of the WO 2014/068079 publication was identified as an advantageous property, as this region showed a significant overlap with the binding sites of BAFF and APRIL, the two natural ligands of CD269. No anti-CD269 antibody described in the art previously had shown such comprehensive overlap with the BAFF and APRIL binding sites.

Certain anti-BCMA antibodies or antibody fragments described herein can bind an epitope comprising one or more of amino acids 13, 15, 16, 17, 18, 19, 20, 22, 23, 26, 27 or 32 of CD269 (BCMA). Optionally, an isolated anti-BCMA antibody or antibody fragment can be characterized in that the antibody binds an epitope consisting of amino acids 13, 15, 16, 17, 18, 19, 20, 22, 23, 26, 27 and 32 of CD269 (BCMA). These residues represent the amino acids that interact directly with the anti-BCMA antibody, as identified by the crystal structure data shown in WO 2014/068079. The numbering of these residues was carried out with respect to the N-terminal sequence of CD269.

In certain embodiments, the anti-BCMA antibody binds CD269 (BCMA) and disrupts the BAFF-CD269 and/or APRIL-CD269 interaction. BAFF/APRIL-CD269 interactions are thought to trigger anti-apoptotic and growth signals in the cell, respectively (Mackay, Schneider et al. (2003) Annu Rev Immunol 21:231-264; Bossen and Schneider (2006) Semin Immunol 18:263-275).

Exemplary humanization of anti-BCMA antibody J22.9-xi: J22.9-xi antibody was humanized based on sequence alignment and data obtained from a crystal structure. The sequences of the variable regions were aligned to their respective human homologs using IgBLAST (NCBI). Each proposed mutation was evaluated by visual inspection of the structure before alteration. Binding of the mutants to BCMA can be tested using flow cytometry. The affinity was measured using surface plasmon resonance (ProteOn™ XPR36; Bio-Rad). Preliminary assessment of the binding properties of the humanized sequences showed promising results with respect to their specificity and affinity to the same epitope as described for J22.9-xi binding.

To obtain a BCMA-binding antibody, standard hybridoma technique can be used. E.g., for production of initial anti-BCMA antibody, four (4) BL/6 wild type mice were immunized 6 times with incomplete Freund's adjuvant and 30 μg of the extracellular domain of human BCMA C-terminally fused to Glutathione S-transferase (GST). After cell fusion followed by a screening period, the J22.9 hybridoma was shown to secrete an anti-BCMA antibody.

### Linkers

Any number of art-recognized linker moieties can be used to join anti-BCMA antibodies with nanoparticles possessing enhanced imaging characteristics, thereby forming anti-BCMA antibody-nanoparticle compositions within the scope of the conjugates described herein. In exemplary embodiments, the reactive amine groups on the surface of compositions present heterobifunctional linker molecules, (e.g., “anchoring points”) via an N-hydroxysuccinimide ester (e.g., NHS) reaction with amine groups. In some embodiments, the heterobifunctional anchoring linker (e.g., a bifunctional PEG macromer) may include the amine-reactive NHS ester on one end, a short (e.g., approximately 2 kilo daltons (kDa)) PEG chain, and an acrylate group on the other end. In certain embodiments, the heterobifunctional linker (e.g., a bifunctional PEG macromer) may include the amine-reactive NHS ester on one end, the short PEG chain, and a thiol group on the other end. The short PEG linker also provides additional degrees of freedom to the acrylate group or the thiol group at the end, making it easier to link to the hydrogel coating in the second reaction. In specific embodiments of the instant disclosure, a bissulfosuccinimidylsuberate (BS3) linker is used for conjugation of NP to anti-BCMA antibody. Alternative linkers—e.g., ones possessing more directed functionality than certain NHS-NHS homobifunctional linkers described herein—are also expressly contemplated.

It is contemplated that conjugation of NP to antibody can be performed in a number of ways, including use of an external linker to conjugate to the NP to create a link to the antibody (where two different functionalities can be selected and mixed together in the same linker, e.g., NHS linker (reactive towards amines) or maleimide (reactive towards thiols)). A number of other linkers can also be used, including alkyne-azide linkers (reacted via copper-catalyzed click chemistry), cyclooctyne-azide (copper-less click chemistry), TCO-tetrazine, etc. Since NPs of the disclosure possess amines, one end of the linker will tend to be NHS, but the composition of the other end of the linker can vary depending upon the antibody handle. Thiol-decorated/functionalized antibodies create scenarios where NHS-maleimide linkers and/or NHS-maleimidocaproyi linkers can be employed with good effect. Additionally and/or alternatively, extra arms can be created upon the polymer itself, thereby creating free amines with a thiol group, which can directly conjugate the NP to the antibody without requiring a linker to bridge the two moieties (NP and antibody).

### Nanoparticles

Nanoparticles of uniform size and shape (e.g., 3-5 nm diameter) have been proven an effective tool for bioimaging. Nanoparticles have a high area-to-volume ratio; they are very reactive, good catalysts and adhere to biological molecules. One nanoparticle material is silicon as it is inert, non-toxic, abundant and economic. The silicon surface can be functionalized. Silicon nanoparticles show efficient photoluminescence in the visible part of the electromagnetic spectrum and are bioinert and chemically stable. One material which has similar biocompatibility is porous silicon. Particles smaller than 100 nm show an enhanced permeability and retaining effect (EPR effect) in tumours, an important nonspecific targeting effect. Silicon nanoparticles, also known as silicon quantum dots, can be used in imaging technologies but also for LED, photovoltaics, lithium ion batteries, transistors, polymers or two-photon absorption.

A number of nanoparticles can be used in the conjugate compositions of the current disclosures, including the exemplified silica-based gadolinium NPs as described herein and, e.g., polymer NPs such as those disclosed in U.S. Pat. No. 9,381,253 (polymer brush nanoparticle for organic MRI contrast) and an exemplary polymer nanoparticle for in vivo CRISPR modification (as described in WO 2017/004509).

### Magnetic Resonance Imaging (MRI)

MRI is one of the most used techniques for medical diagnostics, combining the advantages of being non-invasive, quick and without danger for the patient. It is based on observation of the relaxation of the protons of water, which is directly dependent on magnetic fields (the important magnetic field BO and radio-frequency fields), pulse sequence, the environment of the water in the organism, etc. Interpretation of the MRI images then gives access to identification of most tissues. The contrast can be increased by two types of agents: positive T1 and negative T2 contrast agents. Positive contrast agents, i.e. T1, which permit lightening of the image as contact of water with the contrast agent makes it possible to reduce the longitudinal relaxation time: T1. Gd(III)DTPA or Gd(III)DOTA are examples of T1 contrast agents used in clinical practice and contemplated/employed within the instant disclosure.

Certain nanoparticles known in the field and as employed herein are useful in particular as contrast agents in imaging (e.g., MRI) and/or in other diagnostic techniques and/or as therapeutic agents, which give better performance than known nanoparticles of the same type and which combine both a small size (for example less than 20 nm) and a high loading with metals (e.g., rare earths), in particular so as to have, in imaging (e.g., MRI), strong intensification and a correct response (increased relaxivity) at high frequencies.

Exemplary nanoparticles according to the disclosure, possessing a diameter dl between 1 and 20 nm, can each comprise a polyorganosiloxane (POS) matrix including gadolinium cations optionally associated with doping cations; a chelating graft C1 DTPABA (diethylenetriaminepentaacetic acid bisanhydride) bound to the POS matrix by an —Si—C— covalent bond, and present in sufficient quantity to be able to complex all the gadolinium cations; and optionally another functionalizing graft Gf* bound to the POS matrix by an —Si—C— covalent bond (where Gf* can be derived from a hydrophilic compound (PEG); from a compound having an active ingredient PA1; from a targeting compound; and/or from a luminescent compound (fluorescein)).

### Administration

A nanoparticle-anti-BCMA antibody conjugate of the instant disclosure may be administered via a number of routes of administration, including but not limited to: subcutaneous, intravenous, intrathecal, intramuscular, intranasal, oral, transepidermal, parenteral, by inhalation, or intracerebroventricular.

The term “injection” or “injectable” as used herein refers to a bolus injection (administration of a discrete amount of an agent for raising its concentration in a bodily fluid), slow bolus injection over several minutes, or prolonged infusion, or several consecutive injections/infusions that are given at spaced apart intervals.

In some embodiments of the present disclosure, a formulation as herein defined is administered to the subject by bolus administration.

The nanoparticle conjugate is administered to the subject in an amount sufficient to achieve concentrations at the desired site of imaging (and/or treatment, e.g., where a drug or other agent is administered) determined by a skilled clinician to be effective, for example in an amount sufficient to achieve concentrations in the vicinity of from about 1×10⁻⁸ to about 1×10⁻¹ moles/liter. In some embodiments of the invention, the nanoparticle conjugate is administered at least once a year. In other embodiments of the invention, the nanoparticle conjugate is administered at least once a day. In other embodiments of the invention, the nanoparticle conjugate is administered at least once a week. In some embodiments of the invention, the nanoparticle conjugate is administered at least once a month.

Exemplary doses for administration of a nanoparticle conjugate of the disclosure to a subject include, but are not limited to, the following: 1-20 mg/kg/day, 2-15 mg/kg/day, 5-12 mg/kg/day, 10 mg/kg/day, 1-500 mg/kg/day, 2-250 mg/kg/day, 5-150 mg/kg/day, 20-125 mg/kg/day, 50-120 mg/kg/day, 100 mg/kg/day, at least 10 ug/kg/day, at least 100 ug/kg/day, at least 250 ug/kg/day, at least 500 ug/kg/day, at least 1 mg/kg/day, at least 2 mg/kg/day, at least 5 mg/kg/day, at least 10 mg/kg/day, at least 20 mg/kg/day, at least 50 mg/kg/day, at least 75 mg/kg/day, at least 100 mg/kg/day, at least 200 mg/kg/day, at least 500 mg/kg/day, at least 1 g/kg/day, and an imaging and/or therapeutically effective dose that is less than 500 mg/kg/day, less than 200 mg/kg/day, less than 100 mg/kg/day, less than 50 mg/kg/day, less than 20 mg/kg/day, less than 10 mg/kg/day, less than 5 mg/kg/day, less than 2 mg/kg/day, less than 1 mg/kg/day, less than 500 ug/kg/day, and less than 500 ug/kg/day.

In some embodiments of the invention, a therapeutic agent distinct from the nanoparticle conjugate is administered prior to, in combination with, at the same time, or after administration of the imaging and/or therapeutically effective amount of a nanoparticle conjugate of the disclosure. In some embodiments, the second therapeutic agent is selected from the group consisting of a chemotherapeutic, an antioxidant, an antiinflammatory agent, an antimicrobial, a steroid, etc.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988. Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rero), (4th Ed., Univ. of Oregon Press, Eugene, 2000).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

The present invention is described by reference to the following Examples, which are offered by way of illustration and are not intended to limit the invention in any manner. Standard techniques well known in the art or the techniques specifically described below were utilized.

### Example 1: Materials and Methods

### Cell Lines

The human MM cell line MM.1S was purchased from ATCC (Manassas, Va., USA). The MM.1S GFP⁺ Luc⁺ cell line was generated by retroviral transduction, using the pGC-GFP/Luc vector. Cells were authenticated by short tandem repeat DNA profiling. MM.1S, OPM2 and KMS11 cells were cultured in RPMI media (e.g., RPMI-1640 media; Sigma, USA) supplemented with 10% fetal bovine serum (Sigma, USA), 1% penicillin-streptomycin (Invitrogen, USA) and 1% glutamine (Invitrogen, USA). Optimal conditions of 37° C. and 5% CO₂ were maintained in a humidified incubator.

Silica-Based Gadolinium Nanoparticle Synthesis. Including Synthesis of Antibody-Conjugated Gadolinium-Based Nanoparticles (Gd—NPs)

Ultra-small, silica-comprised Gd—NPs were provided by NH Theraguix, Inc. (Villeurbanne, France) and were synthesized following previously reported procedures (Detappe et al. J Control Release 238: 103-113; Detappe et al. Sci Rep 6: 34040). Such nanoparticles are also described, e.g., in US 2013/0195766

The NP constructs were conjugated with mouse-anti-human SLAMF7 and BCMA monoclonal antibodies (Biolegend Inc., San Diego, Calif.), using a previously reported homobifunctional linker chemistry (Schmidt and Robinson. Nat Protoc 9: 2224-2236). Briefly, Gd—NPs were diluted in UltraPure water to a final concentration of 50 nM. A 1:10 molar ratio of bissulfosuccinimidylsuberate (BS3) linker was mixed with Gd—NPs for 30 min and at room temperature to promote the generation of linker-bound nanoparticles. These surface-modified Gd—NPs were then combined with the monoclonal antibodies at a 1:100 molar ratio; and, the suspensions were stirred for 1 h at room temperature. The nanoparticle-antibody complexes were purified by centrifugation filtration, using a filtration device equipped with a 50 kDa molecular weight cutoff membrane (Milipore) that was spun at 5,000 r.p.m.; centrifugation concentration was subsequently followed by resuspension of the nanoparticle-antibody complexes in 1M PBS. This process was conducted in triplicate to assure removal of all excess free antibodies into the filtrate and to concentrate the suspensions of pure NP-SLAMF7 and NP-BCMA. The final concentrations of the nanoparticle-antibody complexes were determined by ICP-MS, using an Agilent 7900 (Agilent Technologies, Inc., Santa Clara, Calif.).

### In Vitro Assays Determining the Specificity of Nanoparticle-Antibody Complexes to Bind MM Cells

Flow cytometry analyses of MM cell lines treated with various nanoparticle-antibody complexes were performed. The cells were first mixed with suspensions of Gd—NP, NP-SLAMF7 or NP-BCMA (0.5 mM) for 30 min, washed with fresh media, and resuspended in solution (1×10⁶ cells/mL). The treated cells were then incubated with PerCP/Cy5.5-labeled anti-human BCMA antibodies at 37° C. and for one hour, which served as a competitive label to NP-BCMA (whose binding decreased fluorescent labeling with this reagent). Populations of Cy5.5-labeled cells were subsequently detected by flow cytometry. To cross-validate the results, ICP-MS was utilized to quantify the amounts of Gd bound per cell. To perform these later experiments, the treated MM cell lines were lysed with 0.3% Triton-X 100 solution prior to precise enumeration of the amounts of Gd in each sample, using ICP-MS. As a third method of validating the binding of nanoparticle-antibody complexes to MM cells, confocal microscopy was performed to visualize the colocalization of fluorescently-labeled antibodies with nanoparticle-antibody complexes, which had been labeled with a separate fluorophore, on the surfaces of MM cells. For these experiments, Gd—NPs were first conjugated with Cy5-NHS at 1:1000 molar ratio of fluorophore to nanoparticle, using EDC/NHS chemistry. The monoclonal antibodies were similarly labeled with AF488-NHS (1:1000 molar ratio of fluorophore to antibody) prior to nanoparticle conjugation as previously described (vide supra). MM cell lines were incubated with the dual-fluorophore conjugated nanoparticle-antibody complexes for 30 min, fixed in iced-cold methanol, and mounted on cover slips coated with Dapi Fluoromount-G (SouthernBiotech). Confocal microscopy (Olympus FV12000, Olympus) then proceeded to verify co-location of the two fluorophores in a punctate distribution on cellular surfaces.

### Animal Model

GFP⁺/Luc⁺ MM1.S cells were administered to SCID/beige mice (5×10⁶ cells/mouse; n=5 mice per group) via IV dissemination, establishing an orthotopic murine xenograft model of MM. Tumor growth was monitored weekly by bioluminescence imaging (BLI), using an IVIS Spectrum-bioluminescent and fluorescent imaging system (Perkins Elmer). A murine model of MRD was further established by treating these mice with Bortezomib (0.5 mg/kg daily×3 doses) followed by Melphalan (5.5 mg/kg×1 dose).

### Imaging Studies

MR image acquisition was conducted with a preclinical 7-Tesla BioSpec 70/20 MRI scanner (Bruker BioSpin, Billerica, Mass.). A dose equivalent of 0.25 mg/g of Gd—NPs conjugated to 80 μg/mL of anti-BCMA antibodies were administered by IV injection into each mouse prior to imaging. A T1 GRE sequence employing a repetition time of 87 ms, echo time of 3.9 ms, and a flip angle of 60° was utilized for imaging. The acquisition matrix size and reconstructed matrix was 256×256 pixels; the slice thickness was 5 mm. When comparing imaging parameters obtained with the different Gd-based contrast agents, MRI was performed at various time intervals after contrast administration; and, the results were compared to baseline images. For the early diagnostic and MRD quantification studies, MRI was performed 30 min post-IV injection. CT acquisitions were conducted on a preclinical Inveon CT scanner (Siemens) equipped with a 50 kVp source; the image resolution was 10.2 pixels/mm; and, a slice thickness of 0.1 mm was utilized. CT imaging was performed at various time intervals and before the injection of each MR contrast agent in order to compare changes in the SNR for different disease burdens detected via each imaging modality (vide infra).

### Quantitative Comparisons of Imaging Modalities

Evaluation of the relative detection sensitivity for plasma cells at different time points and/or via different imaging modalities was performed by conducting a signal-noise-ratio (SNR) calculation on each acquired image. These SNR values were obtained after first performing a 3D segmentation of the spine and a femur of each animal, using Fiji freeware (https://fiji.sc/). Each image was normalized to the same intensity level and a region of interest (ROI), including the whole examined organ (i.e. spine or femur), was segmented; the signal intensity in the ROI was recorded and compared to the background level, which was measured on each scan. SNR and normalized SNR values were calculated according to equations (1) and (2): (1) SNR=intensity/noise; (2) Normalized SNR(i)=SNR(i)/SNR_{baseline}. Absolute quantification of the uptake of various Gd-based contrast agents was determined, using ICP-MS (Agilent 7900) and by following previously described protocols. Briefly, animals were sacrificed at 30 min after contrast injection; their excised organs were dissolved in a 70% HCl solution; and, the Gd content of each organ was determined.

### Lambda Light-Chain Quantification

Mice were bled once per week and immediately before imaging. Serum was separated from blood samples and frozen at −80° C. until the end of the study. Serum samples were diluted 1:10 v:v with PBS and a clinical-grade immunoassay, which is routinely performed in the pathology core of the Brigham and Women's Hospital (Boston, Mass.), was used to quantify the amounts of lambda light chains present in each sample.

### Receiver Operator Characteristic Comparison

The ROC curve was used to represent the ability of the SNR to discriminate the presence or absence of tumor cells. The SNR at 5 weeks post-tumor cell implantation was enumerated for each of the various imaging modalities and served as a metric by which to compare their detection sensitivities. The class was defined for each time point using the following method: baseline measurements prior to tumor cell implantation served as the control (Ct₀=0) and were compared against subsequent time points (Cₜ=1) with the assumption that tumor cells were thereafter always present. To ensure that the prediction was not random, a two-sided Wilcoxon rank-sum test was employed. A p-value below 0.05 indicates that the SNR value for a given class was significantly different than that of another class.

### Statistical Analyses

All in vitro statistical analyses were performed using GraphPad Prism software (V.7.1). The ability to discriminate the presence of MRD using each of the different medical imaging techniques was performed using R version 3.3.3.

### Example 2: Development of Antibody-Conjugated, Ultra-Small, Gadolinum-Based Nanoparticles: NP-Anti-BCMA Conjugates Detected MM Presence and Progression in Cell Lines and in Mice

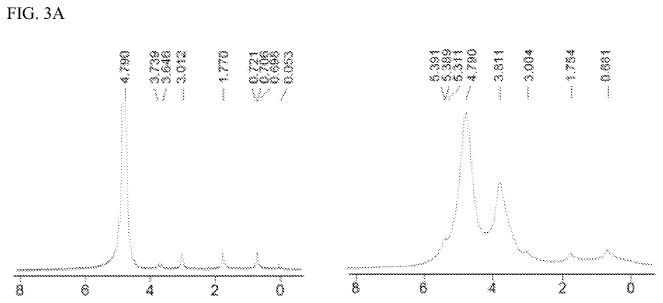

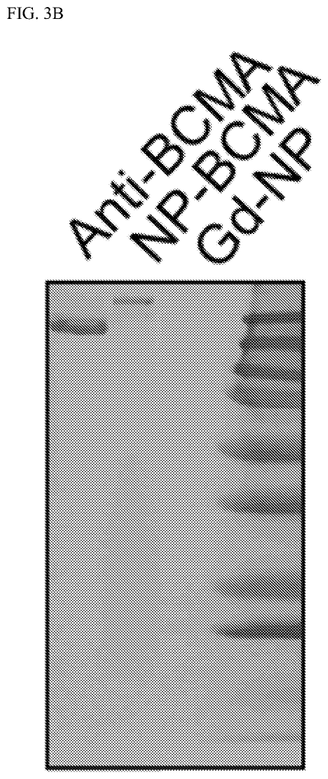

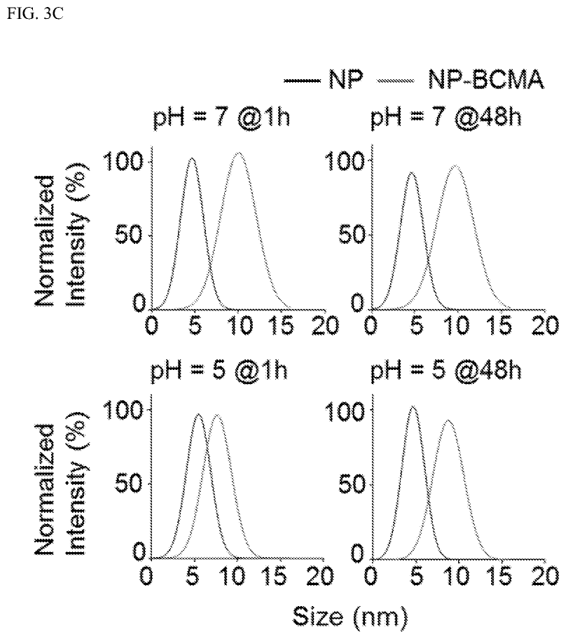

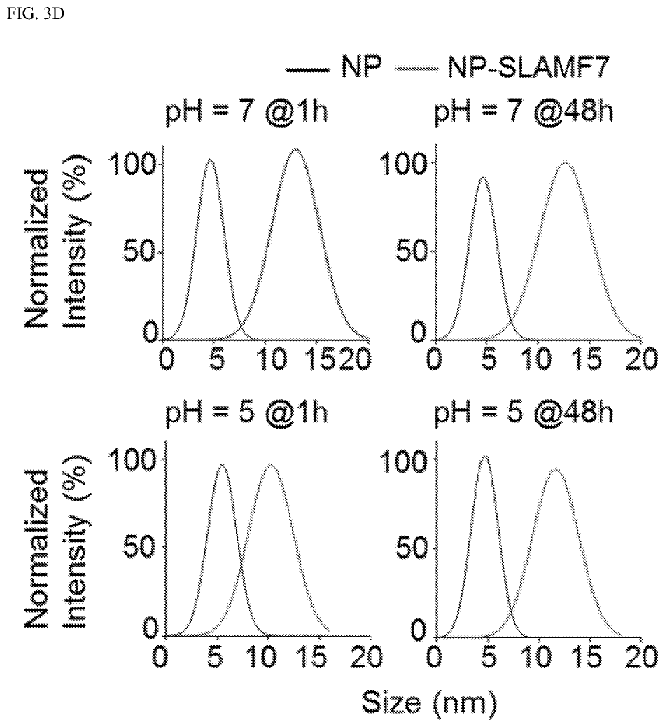

As shown in FIG. 1A, BCMA levels increased with the advancement of MM progression, making BCMA an attractive cell-surface receptor biomarker useful for monitoring MM progression, response to therapeutics and/or MRD status. The above-described conjugate of a sub-5 nm silica-based gadolinium NP and anti-BCMA monoclonal antibody (or, alternatively, a conjugate of the sub-5 nm silica-based gadolinium NP and anti-SLAMF7 monoclonal antibody) was designed such that the NP core decorated with free N-hydroxysuccinimide (NHS) groups was conjugated to NHS groups on the surface of the antibodies via a bissulfosuccinimidyl suberate crosslinker (FIG. 1B). Both SLAMF7 and BCMA antigens are highly expressed and almost exclusively present on the surfaces of B-cells (Lonial et al. N Engl J Med 373: 621-631; Novak et al. Blood 103: 689-694). BCMA further plays an important role in plasma cell transformation and MM progression (Nutt et al. Nat Rev Immunol 15: 160-171; FIG. 1A), making it an attractive and specific biomarker for MRD detection. As noted above, to generate the MM-targeted contrast agent of the instant disclosure, the surfaces of employed Gd—NPs were decorated with free NHS groups and were conjugated to NHS-modified amino groups on anti-SLAMF7 and BCMA antibodies via a bissulfosuccinimidyl suberate crosslinker (FIG. 1B). The targeting efficiencies of such nanoparticle-antibody complexes were subsequently evaluated, both in vitro and in vivo, prior to performing a comparative study to determine their detection capabilities for MRD with respect to other diagnostic modalities. Chemical coupling of mouse anti-human SLAMF7 and BCMA antibodies to Gd—NPs, generating NP-SLAMF7 and NP-BCMA constructs, respectively, were performed using EDC/NHS chemistry. Conjugation of the antibodies to the NP was validated for each NP-antibody construct by high-performance liquid chromatography (HPLC) and by polysaccharide analysis using carbohydrate gel electrophoresis (FIG. 3A and FIG. 3B). As expected, conjugation of the antibodies to the NP increased the size of the respective antibody-NP complexes from 4.4±1.4 nm to 10.01±2.03 nm (NP-BCMA) or 12.9±2.3 nm (NP-SLAMF7) (FIG. 1C). The preceding size results reflect dynamic light scattering (DLS) measurements of average hydrodynamic diameters, which indeed confirmed that both NP-SLAMF7 and NP-BCMA were slightly larger than the unmodified Gd—NPs. These nanoparticle-antibody complexes and their sizes remained stable over time and even under acidic suspension conditions (FIG. 3C and FIG. 3D). A slight decrease of the contrast properties of the NP (as assessed by observation of minor reductions in their relaxivity coefficients as compared to unmodified Gd—NPs) was observed, which, without wishing to be bound by theory, was believed to be due to the positioning of the antibodies at the surface of the gadolinium atoms and therefore the shielding of surface Gd atoms by conjugated antibodies, which resulted in r₁ values that were similar to those of Magnevist™ (e.g., r₁=5.90, 5.49, 5.33, and 4.73 for NP, NP-BCMA, NP-SLAMF7, and Magnevist™, respectively; FIG. 1D).

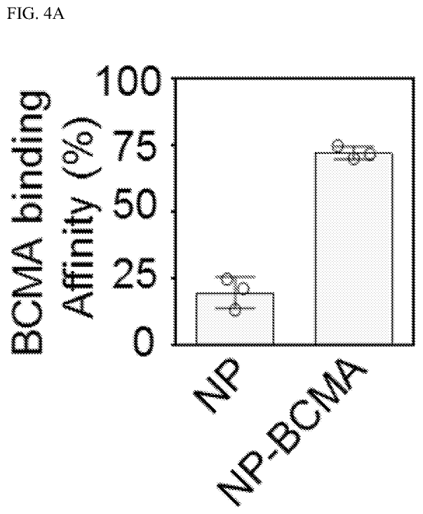

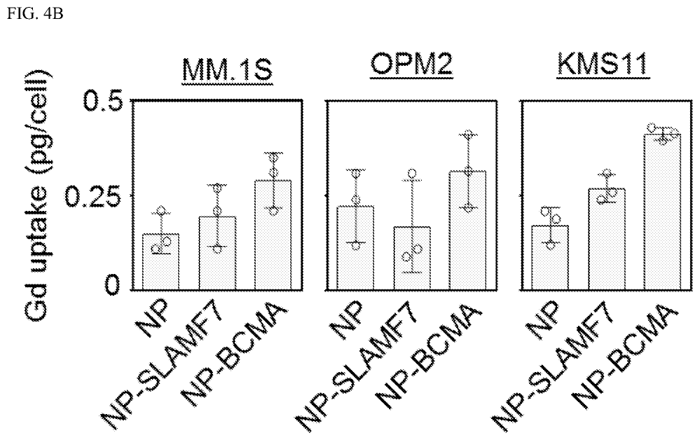

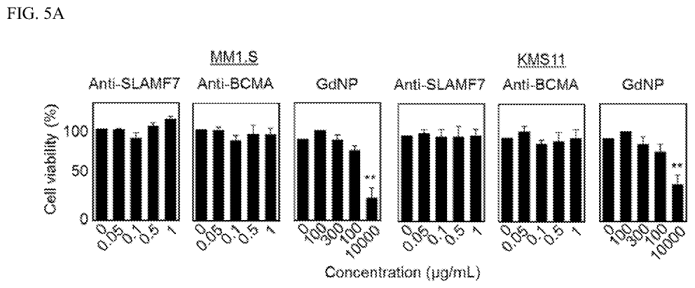

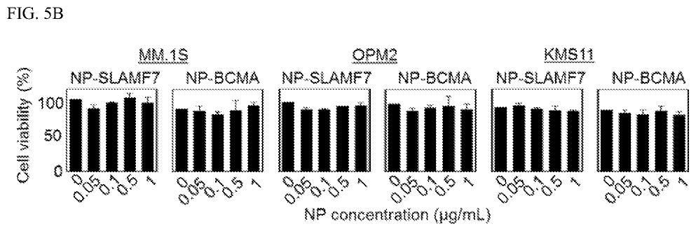

The enhanced in vitro targeting efficiency of the NP-BCMA was subsequently verified by employing a human MM cell line (MM1.S). As shown in FIG. 4A, the NP-BCMA bound 74.1±2.9% of the MM1.S cell surface 30 min post-incubation (as confirmed by flow cytometry analyses that detected cell labeling with NP-BCMA complexes), whereas only 20±4.9% of the cells were bound by the free NP (Gd—NPs) under identical conditions (p<0.001; FIG. 1E, FIG. 4A). The concentration of gadolinium atoms at the surface of the cells (within the final cellular suspensions), as determined by inductively-coupled plasma mass spectrometry (ICP-MS), confirmed a nearly two-fold increase in labeling of three distinct MM cells lines (MM1.S, OPM2, and KMS11) by using NP-BCMA as compared to unmodified Gd—NPs, thereby confirming the efficiency of the cell-surface targeting strategy (FIG. 4B), and fluorescent microscopy of cells that had been incubated with NP-BCMA complexes, where fluorophore-labeled anti-BCMA antibodies were bound to Gd—NPs that had been independently conjugated with a separate fluorescent label, was carried out and confirmed the colocalization of the antibody species and the nanoparticle on the surfaces of MM cells (FIG. 1F). In vitro cellular viability assays of cultured MM cell lines (MM1.S, OPM2, and KMS11) also confirmed that none of the unmodified Gd—NPs, the free antibodies (anti-SLAMF7 or ant-BCMA), nor the nanoparticle-antibody complexes imparted any in vitro toxicities at protein (10,000 μg/mL) and Gd—NP concentrations (1 μg/mL) that were 125- and 4-fold higher, respectively, than those that would be expected in the blood stream after intravenous (IV) administration (FIG. 5A-B).

### Example 3: In Vivo Targeting of Plasma Cells Using Nanoparticle-Antibody Complexes

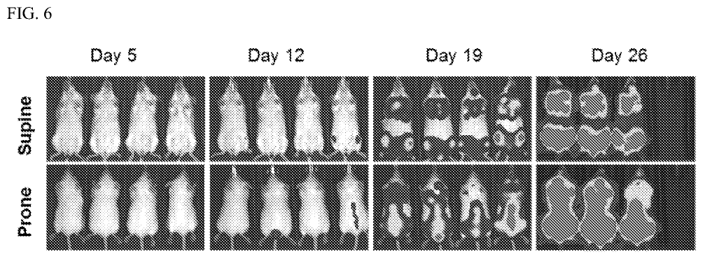

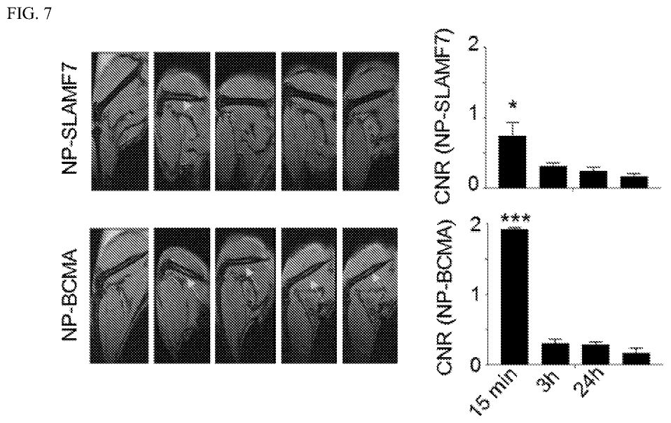

The targeting efficiency of the different NP compositions (NP-SLAMF7 and NP-BCMA, and their ability to detect plasma cells) was then evaluated in a murine model of MM that was established via IV dissemination of MM1.S cells followed by their bone marrow engraftment within immunocompromised SCID-beige mice. Tumor burden (tumor dissemination) was followed by bioluminescence imaging (BLI) at bi-weekly intervals starting on day 19 post-cell (MM1.S) xenotransplantation (injection; FIG. 6). An MRI study was undertaken to compare the efficiencies of the various nanoparticle constructs (Gd—NP, NP-SLAMF7, and NP-BCMA) to identify identical plasma cell burdens and as compared to the FDA-approved contrast agent Magnevist™, a Food and Drug Administration (FDA)-approved contrast agent for MM. Gadolinium (Gd) uptake in the spine and femurs of animals was visualized using a 7T Bruker Biospin MRI scan and by employing a T1-gradiant echo (GRE) sequence (FIG. 1G and FIG. 7).

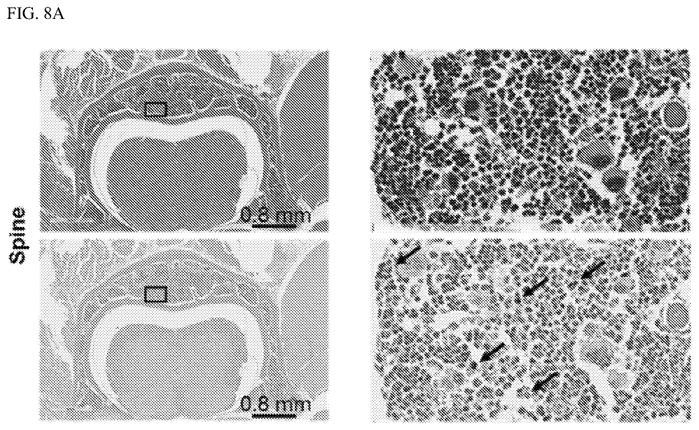

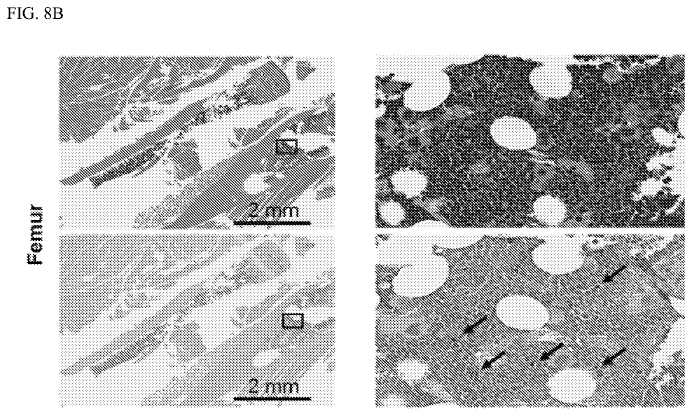

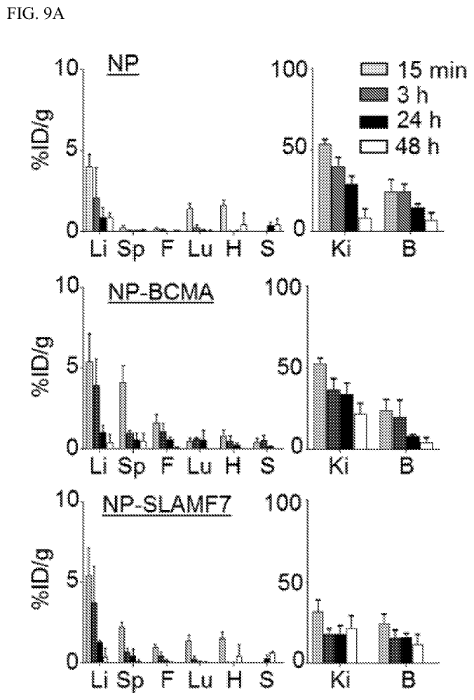

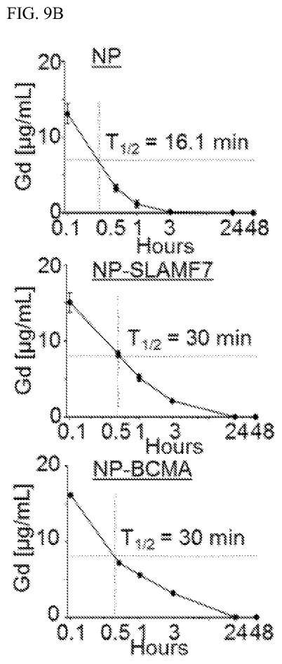

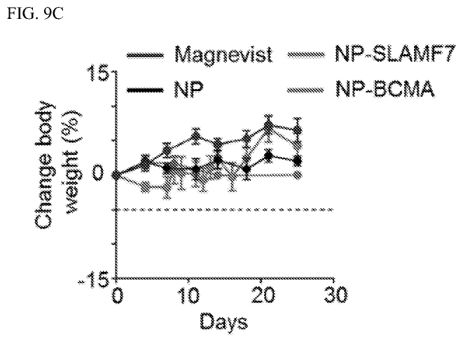

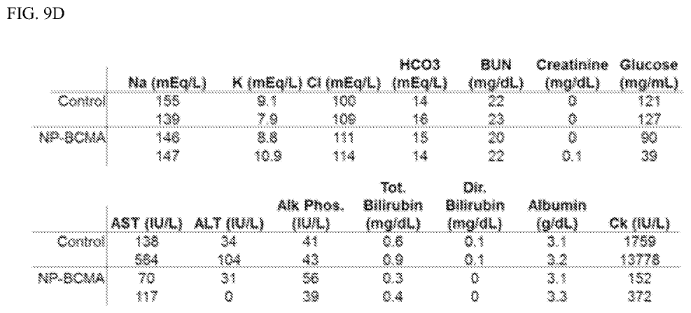

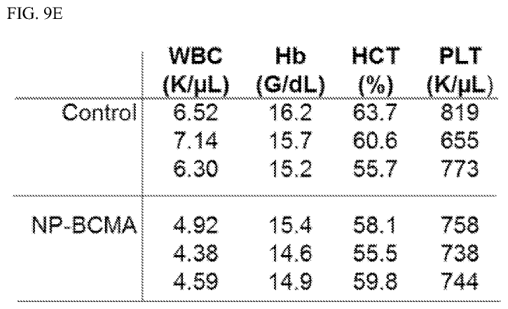

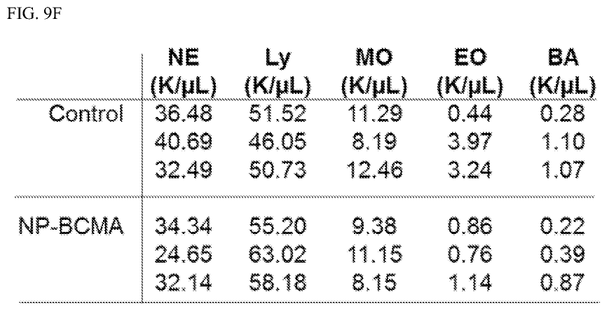

The specificity of each of the administered contrast agents to target MM cells (confirmation of the presence of gadolinium atoms in the tumor region) was confirmed by animal sacrifice immediately after MRI. The femurs and vertebral tissues of each animal were harvested for histologic assessment after staining by H&E and by Prussian blue, which showed sheets of marrow-infiltrating plasma cells labeled with Gd (FIG. 1H, FIG. 8A and FIG. 8B). For quantitative comparisons of MRI sensitivity, the in vivo signal-to-noise ratio (SNR) for the detection of plasma cell populations was enumerated in each image taken at various time points after the administration of different Gd-based contrast agents; signal intensities were quantified after a 3D segmentation of the spines and femurs of the animals (FIG. 1I; FIG. 9A to FIG. 9F). Specifically, signal intensity was quantified after a 3D segmentation of the spine and femurs. The SNR quantification demonstrated the enhanced sensitivity of NP-BCMA and NP-SLAMF7 conjugates, as compared to passive targeting agents Gd—NP and Magnevist™, to detect plasma cell populations. As soon as 30 min post-i.v. injection of the nanoparticle-antibody complexes, animals that had been administered NP-SLAMF7 demonstrated an ˜3.8-fold increase in the SNR for plasmacytomas in the spine while those that had received NP-BCMA exhibited a ˜12-fold enhancement. Significantly, the NP-BCMA conjugate demonstrated better tumor uptake than NP-SLAMF7 (p=0.0045, one-sided paired t-test), which, without wishing to be bound by theory, was attributed to the greater numbers of surface BCMA antigens per MM cell. No remaining traces of gadolinium were observed in the liver, kidney, lungs, or in other organs at 48 h after administration of any nanoparticle-based contrast agents (Gd—NP, NP-SLAMF7, or NP-BCMA; FIG. 9A).

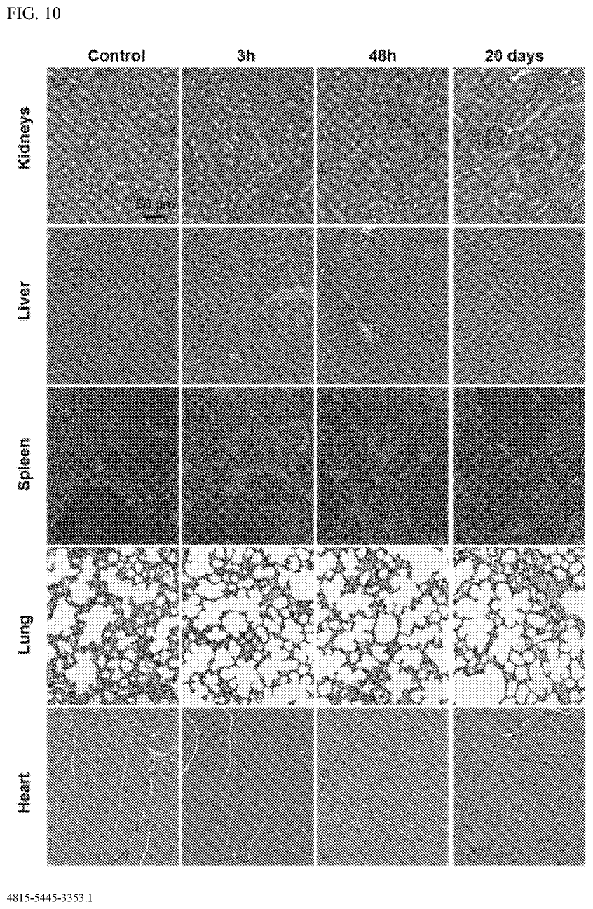

The pharmacokinetic profiles of NP-SLAMF7 and NP-BCMA were similar (FIG. 9B) their circulatory half-lives were longer than that of the unmodified Gd—NPs (t_{1/2}=16.1, 25.2 and 30.3 min for Gd—NP, NP-SLAMF7 and NP-BCMA, respectively). This enhancement in vascular persistence, without wishing to be bound by theory, was attributed to their slightly larger sizes and to the intrinsic properties of the selected antibodies. NP-SLAMF7 and NP-BCMA were found to exhibit rapid renal clearance (presumably because even the NP-antibody conjugates of the current disclosure possessed sizes lower than 15 nm), which limited their long-term exposure to healthy organs (thereby limiting the long-term contact of the gadolinium with healthy organs). The constructs were well tolerated by BALB/c mice, as evidenced by stable animal weights over a two-week period after a single dose IV administration (FIG. 9C, where no decrease in body weight was observed). Terminal blood studies confirmed normal basic metabolic panels (BMPs; FIG. 9D), complete blood counts (CBCs; FIG. 9E, where no difference was observed), and white blood cell differential counts (FIG. 9F, where the chemistry panel was unchanged) at the end of this observation period. H&E staining of excised tissues also demonstrated no evidence of microarchitectural distortion (FIG. 10, where no difference was observed). As such, the nanoparticle-antibody complexes were deemed to exhibit no acute toxicities. ICP-MS of excised organs taken from MM1.S tumor-bearing SCID-beige mice sacrificed at various time points confirmed that 4.2±0.4% of the injected dose per gram (ID/g) localized to plasma cells in the spine while 2.01±0.1% ID/g was found in plasma cells in the femurs at 30 min post-injection (FIG. 1I and FIG. 1J).

### Example 4: Comparisons of the Sensitivity and Specificity of the BCMA-Targeted Nanoparticle-Antibody Complex with Respect to Conventional Methods for Detecting Minimal Residual Disease Revealed that NP-Anti-BCMA Conjugates Detected MRD in Mammalian Subjects

Possessing a near-ideal targeting efficiency for solid tumors (i.e. as noted above, 4.2±0.4% ID/g in the spine, 2.01±0.1% ID/g in the femur) at 30 min post-injection (as quantified by ICP-MS, the NP-anti-BCMA conjugate was examined to determine if this agent, when combined with a MRI scan, could be employed as an imaging biomarker for the detection of MRD. Comparison of the sensitivity and specificity of NP-BCMA with respect to currently available methods for clinical MRD diagnosis was performed. Significant recent progress in the treatment of MM has been achieved, at least in part attributable to recent growth of in-depth understanding of the MM disease pathogenesis. In particular, therapeutic options for treatment of MM have expanded, e.g., with approval of Elotuzumab and Daratumumab having occurred in 2015. However, even while the survival of MM patients has doubled, it also has been demonstrated that an early treatment of MM patients may increase survival even more. In contrast, it was also demonstrated that at the end of a MM treatment, if a MRD negative status was achieved, the patient had a greater chance of non-relapse, as compared to MRD-positive patients. It was therefore evaluated whether the NP-BCMA conjugate might provide both an early predictor of MM and a MRD biomarker agent.

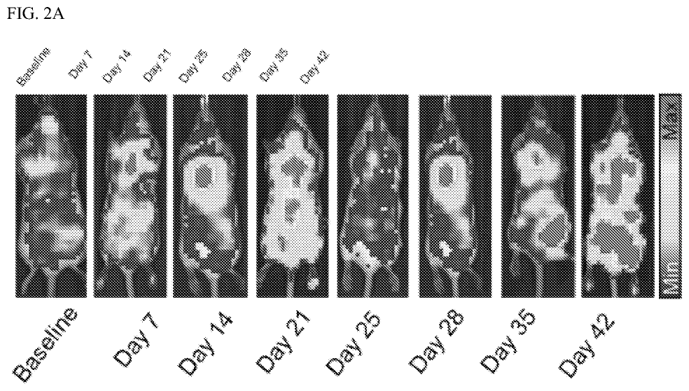

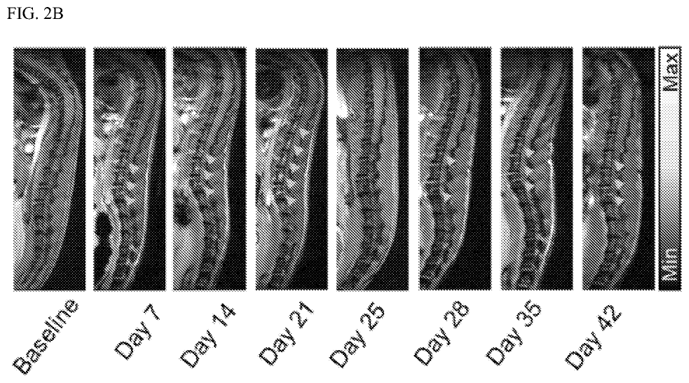

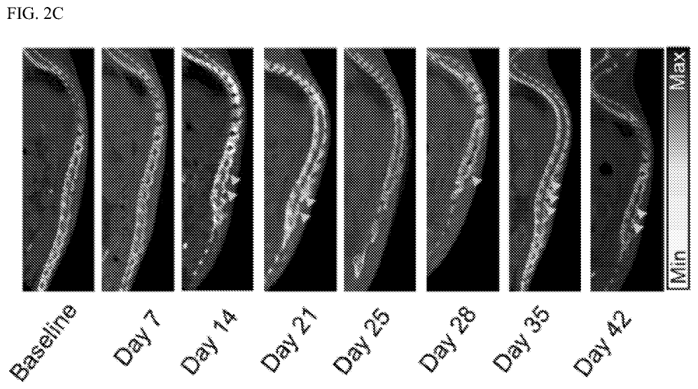

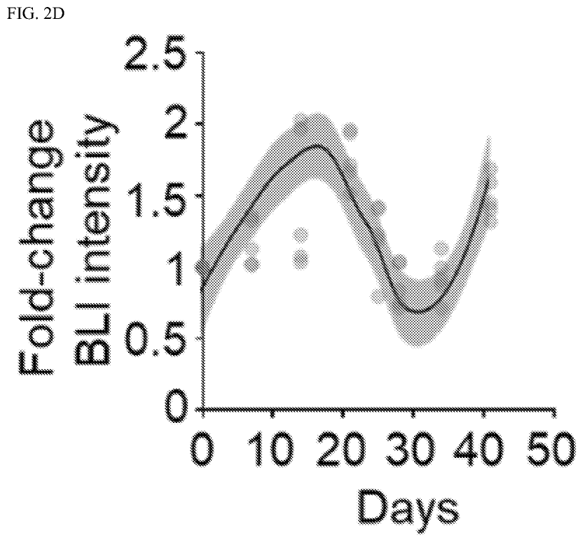

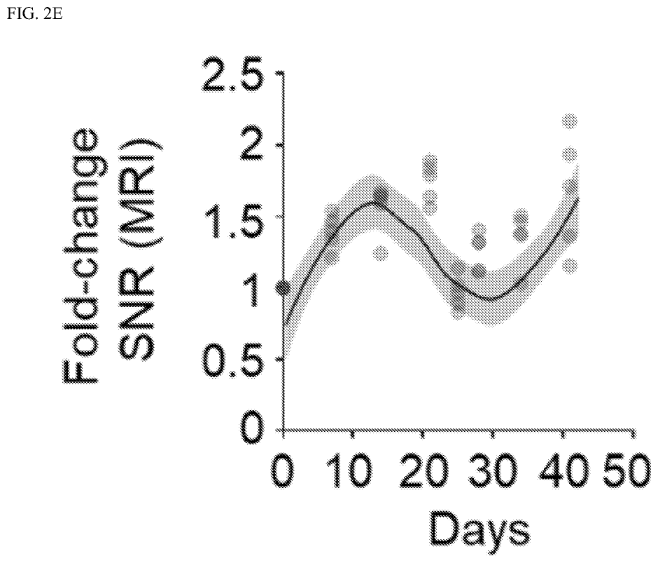

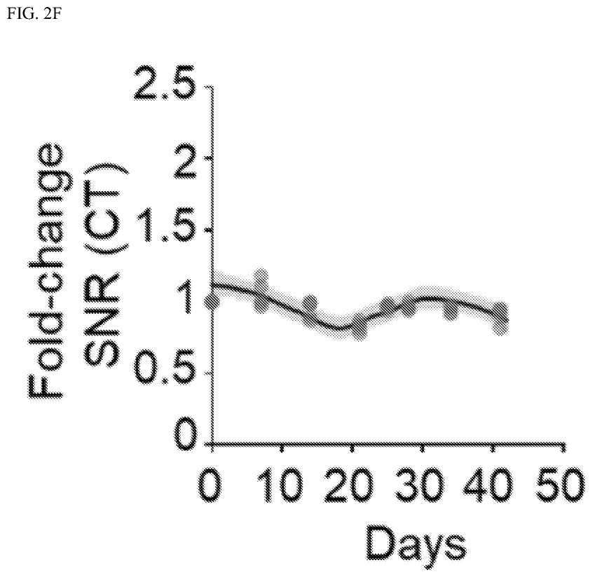

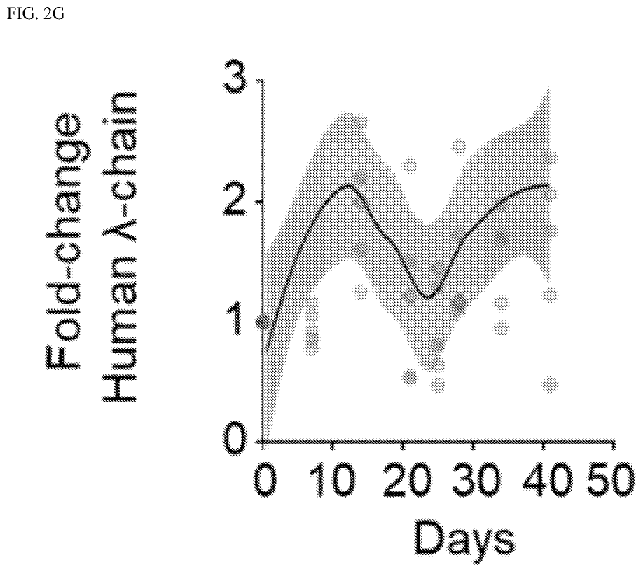

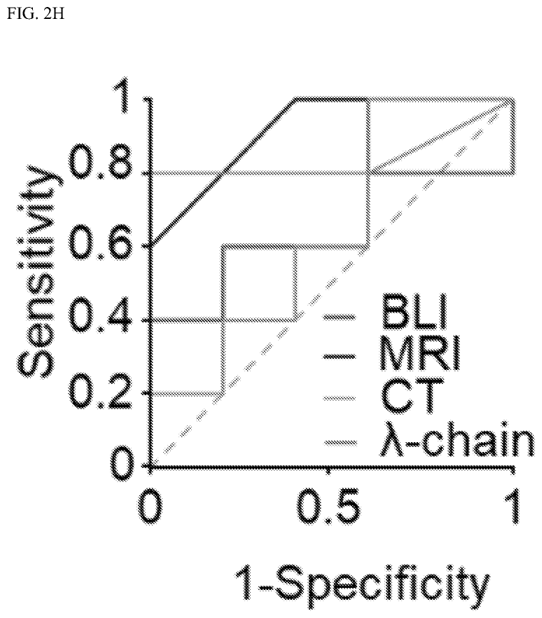

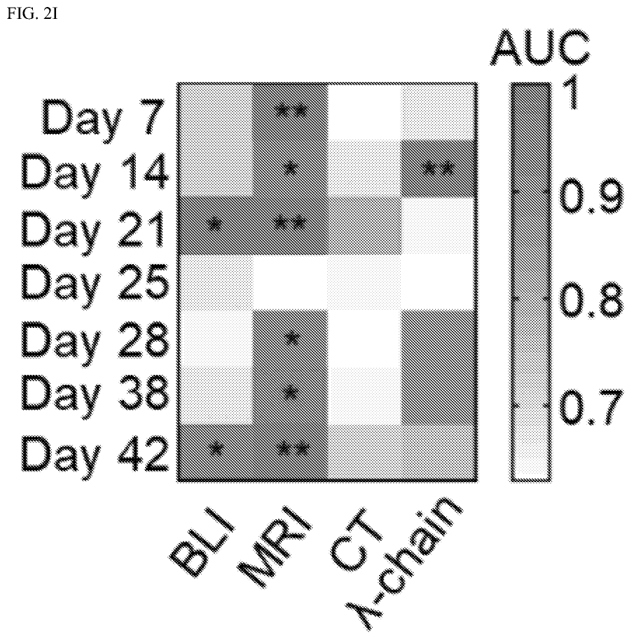

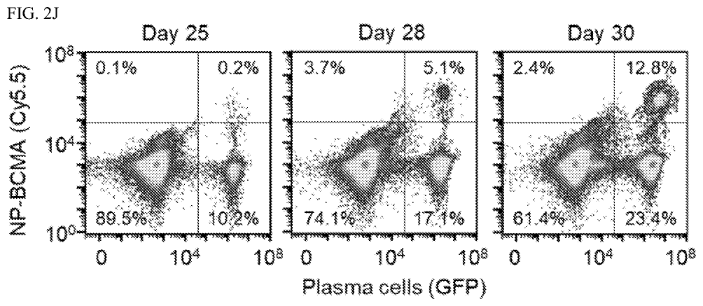

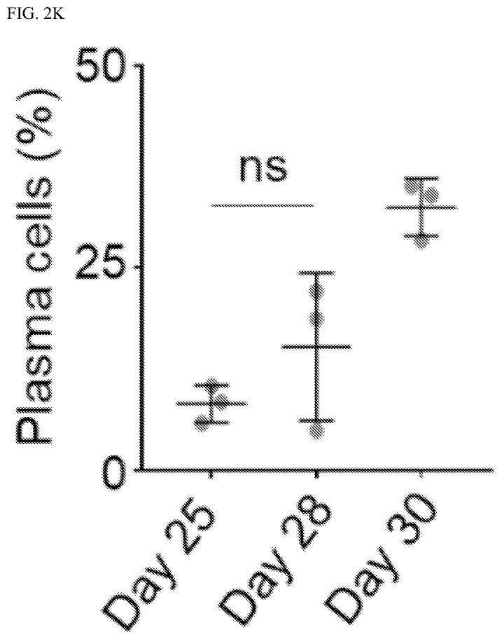

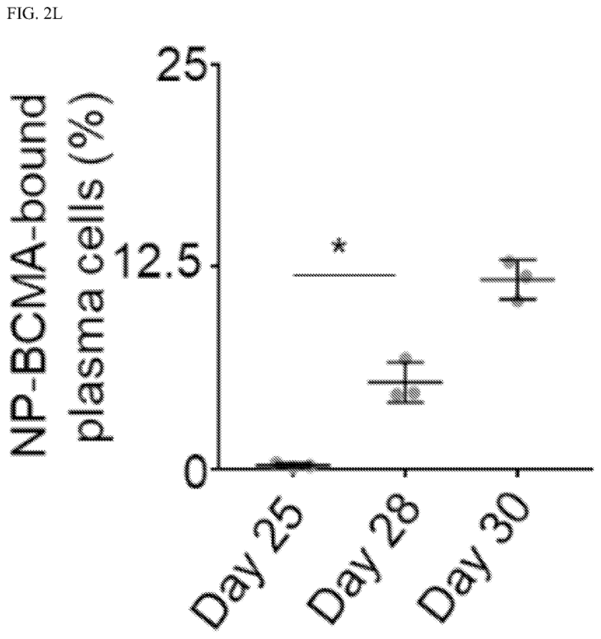

MRD was established as one of the most relevant biomarkers for MM—indeed, most MM patient relapses have been identified as due to the presence of MRD positive signal. It was demonstrated that MRD can be employed to assess the direct therapeutic efficacy of MM therapeutic agents, while also empowering evaluation of future therapeutic decisions. However, detection of MRD is not straightforward. Current techniques to evaluate the presence of a MRD positive status, such as multiparameter flow cytometry and allele-specific oligonucleotide PCR are based on an invasive process, are qualitative, rely on a bone marrow sample, are destructive to samples, and/or are highly time-consuming to administer and evaluate. A common failure in the treatment and imaging of MM is the inability of traditional therapies to reach and combat the bone homing of tumorigenic B-cells. Targeted delivery of effective intracellular agent(s) to target cells has therefore been needed, yet targeted delivery has also presented difficult obstacles. While it has been demonstrated that it is possible to target the bone microenvironment by using bisphosphonate-based nanoparticles that do not possess specific affinities for malignant plasma cells, the current disclosure has identified a new approach to targeting of MM cells specifically. For at least the above reasons, the NP-BCMA conjugate was evaluated as an imaging biomarker for MRD. To do so, a murine model of MRD was established by intravascular dissemination of GFP and luciferase-expressing MM1.S cells (GFP⁺/Luc⁺ MM1.S) followed by therapeutic debulking after 21 days, using three doses of Bortezomib (0.5 mg/kg) and one dose of Melphalan (5.5 mg/kg). Tumor growth was monitored by weekly bioluminescence imaging (BLI), where cell dissemination (MM1.S_{GFP}⁺_{/LUC}⁺) was followed via monitoring once a week by BLI as a gold standard for preclinical monitoring in the tumor cell dissemination method (FIG. 2A), as well as by whole-body MRI (FIG. 2B) and whole body CT scan (FIG. 2C). The MRD model was validated by obtaining a negative BLI signal at day 25 of the treatment, which corresponded to the completion of therapeutic administration(FIG. 2D). Changes in the SNR of the spine over time were subsequently evaluated and used to track disease re-expansion by MRI, which was performed at 30 min after the administration of NP-BCMA at each imaging time point (FIG. 2E); the results were compared to those obtained by CT scan (FIG. 2F). In addition, the increased level of the λ light-chain was followed over time (as a standard method for a patient diagnostic) due to the fact that MM1.s cells are a λ light-chain expresser (MM1.S cells only express the λ light-chain; they do not express the kappa light-chain nor the M-protein (Walker et al. Blood Cancer J 4: e191))—specifically, the levels of serum λ light-chains were measured at the same time points (FIG. 1G).

Results obtained by BLI, MRI, CT, and by the serum λ light-chain assay were compared at 1 week after therapeutic debulking (i.e. 5 weeks after initial tumor cell implantation). A receiver operator characteristic (ROC) curve was generated to assess the sensitivity and specificity of each of the 4 diagnostic modalities to detect the presence of MRD and confirmed the superiority of MRI using NP-BCMA (FIG. 2H). Comparisons of area under the curve (AUC) for the SNR detected by each modality and over the entire duration of the experiment (i.e., from initial tumor cell implantation to therapeutic debulking to eventual animal demise from tumor regrowth) further supported these findings (FIG. 2I). To determine the analytical sensitivity of MRI using NP-BCMA, additional mice were sacrificed on day 25 (immediately after tumor debulking), on day 28 and on day 30 post tumor cell implantation, which corresponded to the first time points after which plasma cells were visible by MRI. Flow cytometry experiments of bone marrow aspirates were conducted (FIG. 2J); the results were enumerated to confirm that MRI using NP-BCMA had a detection threshold for MRD of 2200±450 plasma cells per mouse. As expected, the percentage of plasma cells amongst the total population in the marrow (FIG. 2K), as well as the percentages of NP-BCMA-bound plasma cells (FIG. 2L), increased as a function of time and were due to tumor regrowth.

The specificity and ease of use attributable to the T1 signal enhancement have allowed for a rapid a priori detection of MM disease (including MRD) and can be employed clinically as a predictor, before performing a more in-depth diagnostic of the patient, e.g., performing next generation sequencing. Indeed, results obtained from week 5 (i.e., 1 week post-therapy) (FIG. 2H) and associated area under the curve calculations obtained over the course of the whole experiment (FIG. 2I) have provided the first proof of concept that effective nanoparticle-driven monoclonal antibody can act as an imaging biomarker for MM, and more specifically to predict therapeutic outcomes in a more sensitive and specific fashion than other available imaging modalities and even light chain quantification. Thus, as disclosed herein for MM therapy, the choice of the antigen BCMA as a cell surface receptor for MM and MRD was dictated by its high prevalence and elevated expression levels during MM disease progression from MGUS to SMM. In addition, this marker was also expressed on plasmacytoid dendritic cells, which were promoters of MM cell growth, survival and drug resistance, without BCMA being expressed on naïve and most memory B cells and healthy tissue cells, making this target a unique receptor. Accordingly, NP-BCMA has allowed for the detection of early tumor and extramedullary MM disease, thereby rendering the monitoring of new MM therapeutics via the non-invasive quantification of MRD achievable.

In summary, demonstrated herein is what is believed to be the first proof-of-concept example in which changes in the SNR obtained by serial MRI of ultra-small, Gd-based nanoparticle-antibody complexes have been used as an imaging biomarker to detect MRD. Importantly, the newly disclosed agents described herein were able to circumvent the challenges seen with the first generation of antibody-targeted nanoparticles to achieve precise localization of malignant plasma cells in their natural microenvironment. While they may not be suitable for patients with advanced renal failure, given the well-established risks of all Gd-based contrast agents (Barrett and Parfrey. N Engl J Med 354: 379-386), the constructs disclosed herein may otherwise find utility in prompting early cessation of ineffective therapies and/or therapeutic re-initiation after prolonged periods of MM remission. With the increasing utilization of cell-surface targeted agents in MM therapy (e.g., elotuzumab (Lonial et al. N Engl J Med 373: 621-631), BCMA-targeted chimeric antigen receptor T-cells (Ali et al. Blood 128: 1688-1700; CAR-T), and daratumumab (Lokhorst et al. N Engl J Med 373: 1207-1219)), NP-SLAMF7, NP-BCMA and future formulations of Gd—NP-anti-CD38-antibody complexes may enable MRI to guide patient-specific therapeutic selection. Their successful application may further afford insights into the fabrication of other targeted constructs that may help to translate the full potential of nanomedicine to improve the care and survival of cancer patients.

All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

One skilled in the art would readily appreciate that the present disclosure is well adapted to carry out the objects and obtain the ends and advantages mentioned, as well as those inherent therein. The methods and compositions described herein as presently representative of preferred embodiments are exemplary and are not intended as limitations on the scope of the disclosure. Changes therein and other uses will occur to those skilled in the art, which are encompassed within the spirit of the disclosure, and are defined by the scope of the claims.

In addition, where features or aspects of the disclosure are described in terms of Markush groups or other grouping of alternatives, those skilled in the art will recognize that the disclosure is also thereby described in terms of any individual member or subgroup of members of the Markush group or other group.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Embodiments of this disclosure are described herein, including the best mode known to the inventors for carrying out the disclosed invention. Variations of those embodiments may become apparent to those of ordinary skill in the art upon reading the foregoing description.

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present disclosure provides preferred embodiments, optional features, modification and variation of the concepts herein disclosed may be resorted to by those skilled in the art, and that such modifications and variations are considered to be within the scope of this disclosure as defined by the description and the appended claims.

It will be readily apparent to one skilled in the art that varying substitutions and modifications can be made to the invention disclosed herein without departing from the scope and spirit of the invention. Thus, such additional embodiments are within the scope of the present disclosure and the following claims. The present disclosure teaches one skilled in the art to test various combinations and/or substitutions of chemical modifications described herein toward generating conjugates possessing improved contrast, diagnostic and/or imaging activity. Therefore, the specific embodiments described herein are not limiting and one skilled in the art can readily appreciate that specific combinations of the modifications described herein can be tested without undue experimentation toward identifying conjugates possessing improved contrast, diagnostic and/or imaging activity.

The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context. Those skilled in the art will recognize, or be able to ascertain using no more than routine experimentation, many equivalents to the specific embodiments of the disclosure described herein. Such equivalents are intended to be encompassed by the following claims.

We claim:

1. A targeted nanoparticle conjugate comprising:
a nanoparticle;
a linker; and
an anti-B-cell maturation (BCMA) antibody.

2. The targeted nanoparticle conjugate of claim 1, wherein the nanoparticle of the targeted nanoparticle conjugate is less than 10 nm in size.

3. The targeted nanoparticle conjugate of claim 1, wherein the nanoparticle is a gadolinium nanoparticle, optionally a silica-based gadolinium nanoparticle (SiGdNP).

4. The targeted nanoparticle conjugate of claim 1, wherein the nanoparticle of the targeted nanoparticle conjugate is 30 nm or more in size.

5. The targeted nanoparticle conjugate of claim 1, wherein the nanoparticle is a polymer brush nanoparticle or a nanoparticle comprising clustered regularly interspaced short palindromic repeats (CRISPR) machinery (i.e. sgRNA guides and/or Cas9 mRNA) agents.

6. The targeted nanoparticle conjugate of claim 1, wherein the nanoparticle is a polymer nanoparticle, optionally wherein the targeted nanoparticle conjugate further comprises a drug.

7. The targeted nanoparticle conjugate of claim 1, wherein the nanoparticle is an inorganic nanoparticle.

8. The targeted nanoparticle conjugate of claim 1, wherein the targeted nanoparticle conjugate is approximately 6-15 nm in size, optionally about 8-12 nm in size, optionally wherein the size of the targeted nanoparticle conjugate is stable over time, optionally wherein the size of the targeted nanoparticle conjugate is stable over a period of 15 min or longer, optionally wherein the size of the targeted nanoparticle conjugate is stable over a period of 30 min or longer.

9. The targeted nanoparticle conjugate of claim 1, wherein the targeted nanoparticle conjugate is approximately 15-60 nm in size, optionally about 30-50 nm in size, optionally wherein the size of the targeted nanoparticle conjugate is stable over time, optionally wherein the size of the targeted nanoparticle conjugate is stable over a period of 15 min or longer, optionally wherein the size of the targeted nanoparticle conjugate is stable over a period of 30 min or longer.

10. The targeted nanoparticle conjugate of claim 1, wherein the linker is selected from the group consisting of a N-hydroxysuccinimide (NHS)-to-NHS linker, a NHS-to-haloacetyl, a NHS-maleimide, and a NHS-pyridyldithiol linker.

11. The targeted nanoparticle conjugate of claim 1, wherein the anti-BCMA antibody is a monoclonal antibody or fragment thereof, optionally a human monoclonal antibody or fragment thereof.

12. The targeted nanoparticle conjugate of claim 1, wherein the anti-BCMA antibody is an anti-BCMA antibody fragment, optionally selected from the group consisting of a Fv, a Fab, a Fab′, a Fab′-SH, a F(ab′)₂, a diabody, a linear antibody, a single-chain antibody molecule (e.g., scFv) and a multispecific antibody formed from antibody fragments.

13. The targeted nanoparticle conjugate of claim 1, wherein the anti-BCMA antibody is labeled, optionally wherein the anti-BCMA antibody is labeled with peridinin chlorophyll protein complex (PerCP)/Cy5.5.

14. The targeted nanoparticle conjugate of claim 1, wherein the targeted nanoparticle conjugate comprises a nanoparticle core decorated with free NHS groups, optionally wherein said NHS groups are conjugated on the surface of the anti-BCMA antibody via a bissulfosuccinimidyl suberate crosslinker.

15. The targeted nanoparticle conjugate of claim 1, further comprising a drug moiety, optionally wherein the drug moiety is an anti-CS1 agent or an anti-BCMA agent.

16. A formulation comprising the targeted nanoparticle conjugate of claim 1.

17. The formulation of claim 14, wherein the targeted nanoparticle conjugate is present at a dose equivalent of 0.1-1 mg/g of SiGdNP, optionally at about 0.25 mg/g of SiGdNP.

18. A pharmaceutical composition comprising the targeted nanoparticle conjugate of claim 1 and a pharmaceutically acceptable carrier.

19. A method for detecting the presence and/or localization of multiple myeloma (MM) and/or minimal residual disease (MRD) in a subject, the method comprising:
administering the targeted nanoparticle conjugate of claim 1 to the subject; and
detecting the presence and/or localization of the targeted nanoparticle conjugate in the subject,
thereby detecting the presence and/or localization of MM and/or MRD in the subject.

20. The method of claim 19, wherein the step of administering is performed by injection, optionally by intravenous and/or intraperitoneal injection.

21. The method of claim 19, wherein the step of detecting comprises utilization of a magnetic resonance imaging (MRI) scan.

22. The method of claim 21, wherein the targeted nanoparticle conjugate acts as an imaging biomarker for the detection of MM cells and/or MRD in the subject.

23. The method of claim 22, wherein the targeted nanoparticle conjugate provides contrast that is improved by at least 5-fold, optionally by at least 10-fold, optionally about 12-fold or more as compared to an appropriate non-targeted NP control, optionally wherein a signal-to-noise ratio (SNR) and normalized SNR are calculated according to equations (1) and (2): (1) SNR=intensity/noise; (2) Normalized SNR(i)=SNR(i)/SNR_{baseline}.

24. The method of claim 22, wherein the targeted nanoparticle conjugate possesses a MRI detection threshold for MRD of 100,000 or less plasma cells per subject, optionally 50,000 or less plasma cells per subject, optionally 30,000 or less plasma cells per subject, optionally 20,000 or less plasma cells per subject, optionally 10,000 or less plasma cells per subject, optionally 8,000 or less plasma cells per subject, optionally 6,000 or less plasma cells per subject, optionally 5,000 or less plasma cells per subject, optionally 4,000 or less plasma cells per subject, optionally 3,000 or less plasma cells per subject, optionally about 2,200 plasma cells per subject.

25. The method of claim 19, wherein the step of detecting is performed within approximately 1 hour of the step of administering the targeted nanoparticle conjugate, optionally within approximately 30 minutes of the step of administering the targeted nanoparticle conjugate.

26. The method of claim 19, wherein the targeted nanoparticle conjugate binds approximately 70% of MM cells at 30 minutes after the step of administering the targeted nanoparticle conjugate.

27. The method of claim 19, wherein the targeted nanoparticle conjugate is detected in spine, femur, other bone and/or in the spleen.

28. The method of claim 19, wherein tumor uptake of the targeted nanoparticle conjugate is enhanced relative to an appropriate control non-targeted nanoparticle.

29. The method of claim 19, wherein detecting the presence and/or localization of MM and/or MRD in the subject is used to assess a MM therapy, optionally a therapy comprising administration of an anti-CS1 agent or an anti-BCMA agent, optionally wherein the targeted nanoparticle conjugate is administered in combination with the MM therapy.

30. The method of claim 19, wherein the subject is human.

31. The method of claim 19, wherein the subject is murine.

32. The method of claim 31, wherein the subject is a MRD model mouse, optionally wherein the MRD model mouse is induced by administration of Bortezomib and Melphalan.

33. The method of claim 31, wherein xenograft-derived MM is detected in SCID/beige mice.

34. The method of claim 19, wherein detecting the presence and/or localization of MM and/or MRD in the subject comprises detecting disease progression from MGUS to SMM and/or detecting early tumor and/or extramedullary MM disease.

35. The method of claim 19, wherein the detecting step comprises detecting gadolinium, optionally detecting Gd¹⁵⁵ concentrations.

36. A targeted nanoparticle conjugate comprising:
a nanoparticle comprising multiple sites of conjugation; and
an anti-BCMA antibody.
